SEQUENCE LISTING (109 sequences)

SEQ ID NO: 1 - protein, 638 aa, Homo sapiens
MAGGSATTWGYPVALLLLVATLGLGRWLQPDPGLPGLRHSYDCGIKGMQLLVFPRPGQTLRFKVVDEFGNRFDVNNCSICYHWVTSRPQEPAVFSADYRGCHVLEKDGRFHLRVFMEAVLPNGRVDVAQDATLICPKPDPSRTLDSQLAPPAMFSVSTPQTLSFLPTSGHTSQGSGHAFPSPLDPGHSSVHPTPALPSPGPGPTLATLAQPHWGTLEHWDVNKRDYIGTHLSQEQCQVASGHLPCIVRRTSKEACQQAGCCYDNTREVPCYYGNTATVQCFRDGYFVLVVSQEMALTHRITLANIHLAYAPTSCSPTQHTEAFVVFYFPLTHCGTTMQVAGDQLIYENWLVSGIHIQKGPQGSITRDSTFQLHVRCVFNASDFLPIQASIFPPPSPAPMTQPGPLRLELRIAKDETFSSYYGEDDYPIVRLLREPVHVEVRLLQRTDPNLVLLLHQCWGAPSANPFQQPQWPILSDGCPFKGDSYRTQMVALDGATPFQSHYQRFTVATFALLDSGSQRALRGLVYLFCSTSACHTSGLETCSTACSTGTTRQRRSSGHRNDTARPQDIVSSPGPVGFEDSYGQEPTLGPTDSNGNSSLRPLLWAVLLLPAVALVLGFGVFVGLSQTWAQKLWESNRQ

SEQ ID NO: 2 - protein, 745 aa, Homo sapiens
MACRQRGGSWSPSGWFNAGWSTYRSISLFFALVTSGNSIDVSQLVNPAFPGTVTCDEREITVEFPSSPGTKKWHASVVDPLGLDMPNCTYILDPEKLTLRATYDNCTRRVHGGHQMTIRVMNNSAALRHGAVMYQFFCPAMQVEETQGLSASTICQKDFMSFSLPRVFSGLADDSKGTKVQMGWSIEVGDGARAKTLTLPEAMKEGFSLLIDNHRMTFHVPFNATGVTHYVQGNSHLYMVSLKLTFISPGQKVIFSSQAICAPDPVTCNATHMTLTIPEFPGKLKSVSFENQNIDVSQLHDNGIDLEATNGMKLHFSKTLLKTKLSEKCLLHQFYLASLKLTFLLRPETVSMVIYPECLCESPVSIVTGELCTQDGFMDVEVYSYQTQPALDLGTLRVGNSSCQPVFEAQSQGLVRFHIPLNGCGTRYKFEDDKVVYENEIHALWTDFPPSKISRDSEFRMTVKCSYSRNDMLLNINVESLTPPVASVKLGPFTLILQSYPDNSYQQPYGENEYPLVRFLRQPIYMEVRVLNRDDPNIKLVLDDCWATSTMDPDSFPQWNVVVDGCAYDLDNYQTTFHPVGSSVTHPDHYQRFDMKAFAFVSEAHVLSSLVYFHCSALICNRLSPDSPLCSVTCPVSSRHRRATGATEAEKMTVSLPGPILLLSDDSSFRGVGSSDLKASGSSGEKSRSETGEEVGSRGAMDTKGHKTAGDVGSKAVAAVAAFAGVVATLGFIYYLYEKRTVSNH

SEQ ID NO: 3 - protein, 424 aa, Homo sapiens
MELSYRLFICLLLWGSTELCYPQPLWLLQGGASHPETSVQPVLVECQEATLMVMVSKDLFGTGKLIRAADLTLGPEACEPLVSMDTEDVVRFEVGLHECGNSMQVTDDALVYSTFLLHDPRPVGNLSIVRTNRAEIPIECRYPRQGNVSSQAILPTWLPFRTTVFSEEKLTFSLRLMEENWNAEKRSPTFHLGDAAHLQAEIHTGSHVPLRLFVDHCVATPTPDQNASPYHTIVDFHGCLVDGLTDASSAFKVPRPGPDTLQFTVDVFHFANDSRNMIYITCHLKVTLAEQDPDELNKACSFSKPSNSWFPVEGSADICQCCNKGDCGTPSHSRRQPHVMSQWSRSASRNRRHVTEEADVTVGPLIFLDRRGDHEVEQWALPSDTSVVLLGVGLAVVVSLTLTAVILVLTRRCRTASHPVSASE

SEQ ID NO: 4 - protein, 540 aa, Homo sapiens
MWLLRCVLLCVSLSLAVSGQHKPEAPDYSSVLHCGPWSFQFAVNLNQEATSPPVLIAWDNQGLLHELQNDSDCGTWIRKGPGSSVVLEATYSSCYVTEWDSHYIMPVGVEGAGAAEHKVVTERKLLKCPMDLLARDAPDTDWCDSIPARDRLPCAPSPISRGDCEGLGCCYSSEEVNSCYYGNTVTLHCTREGHFSIAVSRNVTSPPLLLDSVRLALRNDSACNPVMATQAFVLFQFPFTSCGTTRQITGDRAVYENELVATRDVKNGSRGSVTRDSIFRLHVSCSYSVSSNSLPINVQVFTLPPPFPETQPGPLTLELQIAKDKNYGSYYGVGDYPVVKLLRDPIYVEVSILHRTDPYLGLLLQQCWATPSTDPLSQPQWPILVKGCPYIGDNYQTQLIPVQKALDLPFPSHHQRFSIFTFSFVNPTVEKQALRGPVHLHCSVSVCQPAETPSCVVTCPDLSRRRNFDNSSQNTTASVSSKGPMILLQATKDPPEKLRVPVDSKVLWVAGLSGTLILGALLVSYLAVKKQKSCPDQMCQ

SEQ ID NO: 5 - protein, 328 aa, Homo sapiens
QPLWLLQGGASHPETSVQPVLVECQEATLMVMVSKDLFGTGKLIRAADLTLGPEACEPLVSMDTEDVVRFEVGLHECGNSMQVTDDALVYSTFLLHDPRPVGNLSIVRTNRAEIPIECRYPRQGNVSSQAILPTWLPFRTTVFSEEKLTFSLRLMEENWNAEKRSPTFHLGDAAHLQAEIHTGSHVPLRLFVDHCVATPTPDQNASPYHTIVDFHGCLVDGLTDASSAFKVPRPGPDTLQFTVDVFHFANDSRNMIYITCHLKVTLAEQDPDELNKACSFSKPSNSWFPVEGSADICQCCNKGDCGTPSHSRRQPHVMSQWSRSASRN

SEQ ID NO: 6 - protein, 9 aa, Homo sapiens
HPETSVQPV

SEQ ID NO: 7 - protein, 9 aa, Homo sapiens
PETSVQPVL

SEQ ID NO: 8 - protein, 9 aa, Homo sapiens
QEATLMVMV

SEQ ID NO: 9 - protein, 9 aa, Homo sapiens
ATLMVMVSK

SEQ ID NO: 10 - protein, 9 aa, Homo sapiens
LIRAADLTL

SEQ ID NO: 11 - protein, 9 aa, Homo sapiens
TEDVVRFEV

SEQ ID NO: 12 - protein, 9 aa, Homo sapiens
QVTDDALVY

SEQ ID NO: 13 - protein, 9 aa, Homo sapiens
ALVYSTFLL

SEQ ID NO: 14 - protein, 9 aa, Homo sapiens
FLLHDPRPV

SEQ ID NO: 15 - protein, 9 aa, Homo sapiens
RPVGNLSIV

SEQ ID NO: 16 - protein, 9 aa, Homo sapiens
YPRQGNVSS

SEQ ID NO: 17 - protein, 9 aa, Homo sapiens
SSQAILPTW

SEQ ID NO: 18 - protein, 9 aa, Homo sapiens
AILPTWLPF

SEQ ID NO: 19 - protein, 9 aa, Homo sapiens
WLPFRTTVF

SEQ ID NO: 20 - protein, 9 aa, Homo sapiens
VFSEEKLTF

SEQ ID NO: 21 - protein, 9 aa, Homo sapiens
SEEKLTFSL

SEQ ID NO: 22 - protein, 9 aa, Homo sapiens
RLMEENWNA

SEQ ID NO: 23 - protein, 9 aa, Homo sapiens
FHLGDAAHL

SEQ ID NO: 24 - protein, 9 aa, Homo sapiens
IHTGSHVPL

SEQ ID NO: 25 - protein, 9 aa, Homo sapiens
SPYHTIVDF

SEQ ID NO: 26 - protein, 9 aa, Homo sapiens
GLTDASSAF

SEQ ID NO: 27 - protein, 9 aa, Homo sapiens
LTDASSAFK

SEQ ID NO: 28 - protein, 9 aa, Homo sapiens
VPRPGPDTL

SEQ ID NO: 29 - protein, 9 aa, Homo sapiens
RPGPDTLQF

SEQ ID NO: 30 - protein, 9 aa, Homo sapiens
QFTVDVFHF

SEQ ID NO: 31 - protein, 9 aa, Homo sapiens
FTVDVFHFA

SEQ ID NO: 32 - protein, 9 aa, Homo sapiens
ANDSRNMIY

SEQ ID NO: 33 - protein, 9 aa, Homo sapiens
MIYITCHLK

SEQ ID NO: 34 - protein, 9 aa, Homo sapiens
IYITCHLKV

SEQ ID NO: 35 - protein, 9 aa, Homo sapiens
FSKPSNSWF

SEQ ID NO: 36 - protein, 9 aa, Homo sapiens
KPSNSWFPV

SEQ ID NO: 37 - protein, 9 aa, Homo sapiens
RQPHVMSQW

SEQ ID NO: 38 - protein, 9 aa, Homo sapiens
RGDHEVEQW

SEQ ID NO: 39 - protein, 9 aa, Homo sapiens
EATLMVMVS

SEQ ID NO: 40 - protein, 9 aa, Homo sapiens
LMVMVSKDL

SEQ ID NO: 41 - protein, 9 aa, Homo sapiens
MVMVSKDLF

SEQ ID NO: 42 - protein, 9 aa, Homo sapiens
LIRAADLTL

SEQ ID NO: 43 - protein, 9 aa, Homo sapiens
LVMSDTEDV

SEQ ID NO: 44 - protein, 9 aa, Homo sapiens
VSMDTEDVV

SEQ ID NO: 45 - protein, 9 aa, Homo sapiens
VVRFEVGLH

SEQ ID NO: 46 - protein, 9 aa, Homo sapiens
QVTDDALVY

SEQ ID NO: 47 - protein, 9 aa, Homo sapiens
ALVYSTFLL

SEQ ID NO: 48 - protein, 9 aa, Homo sapiens
LVYSTFLLH

SEQ ID NO: 49 - protein, 9 aa, Homo sapiens
FLLDPHRPV

SEQ ID NO: 50 - protein, 9 aa, Homo sapiens
LLHDPRPVG

SEQ ID NO: 51 - protein, 9 aa, Homo sapiens
LSIVRTNRA

SEQ ID NO: 52 - protein, 9 aa, Homo sapiens
IVRTNRAEI

SEQ ID NO: 53 - protein, 9 aa, Homo sapiens
PFRTTVFSE

SEQ ID NO: 54 - protein, 9 aa, Homo sapiens
FRTTVFSEE

SEQ ID NO: 55 - protein, 9 aa, Homo sapiens
TFSLRLMEE

SEQ ID NO: 56 - protein, 9 aa, Homo sapiens
FHLGDAAHL

SEQ ID NO: 57 - protein, 9 aa, Homo sapiens
LRLFVDHCV

SEQ ID NO: 58 - protein, 9 aa, Homo sapiens
LFVDHCVAT

SEQ ID NO: 59 - protein, 9 aa, Homo sapiens
QFTVDVFHF

SEQ ID NO: 60 - protein, 9 aa, Homo sapiens
FANDSNRMI

SEQ ID NO: 61 - protein, 9 aa, Homo sapiens
IYITCHLKV

SEQ ID NO: 62 - protein, 9 aa, Homo sapiens
VMSQWSRSA

SEQ ID NO: 63 - protein, 9 aa, Homo sapiens
WSRSASRNR

SEQ ID NO: 64 - protein, 9 aa, Homo sapiens
LIFLDRRGD

SEQ ID NO: 65 - protein, 9 aa, Homo sapiens
IFLDRRGDH

SEQ ID NO: 66 - protein, 49 aa, Homo sapiens
GGASHPETSVQPVLVECQEATLMVMVSKDLFGTGKLIRAADLTLGPEAC

SEQ ID NO: 67 - protein, 26 aa, Homo sapiens
EACEPLVSMDTEDVVRFEVGLHECGN

SEQ ID NO: 68 - protein, 43 aa, Homo sapiens
CGNSMQVTDDALVYSTFLLHDPRPVGNLSIVRTNRAEIPIECR

SEQ ID NO: 69 - protein, 50 aa, Homo sapiens
IECRYPRQGNVSSQAILPTWLPFRTTVFSEEKLTFSLRLMEENWNAEKRS

SEQ ID NO: 70 - protein, 41 aa, Homo sapiens
KRSPTFHLGDAAHLQAEIHTGSHVPLRLFVDHCVATPTPDQ

SEQ ID NO: 71 - protein, 44 aa, Homo sapiens
DQNASPYHTIVDFHGCLVDGLTDASSAFKVPRPGPDTLQFTVDV

SEQ ID NO: 72 - protein, 34 aa, Homo sapiens
GPDTLQFTVDVFHFANDSRNMIYITCHLKVTLAE

SEQ ID NO: 73 - protein, 20 aa, Homo sapiens
KACSFSKPSNSWFPVEGPAD

SEQ ID NO: 74 - protein, 27 aa, Homo sapiens
SHSRRQPHVMSQWSRSASRNRRHVTEE

SEQ ID NO: 75 - protein, 26 aa, Homo sapiens
ADVTVGPLIFLDRRGDHEVEQWALPS

SEQ ID NO: 76 - protein, 9 aa, Homo sapiens
FLLHDPRPV

SEQ ID NO: 77 - protein, 9 aa, Homo sapiens
RLMEENWNA

SEQ ID NO: 78 - protein, 9 aa, Homo sapiens
ALVYSTFLL

SEQ ID NO: 79 - protein, 9 aa, Homo sapiens
FTVDVFHFA

SEQ ID NO: 80 - protein, 10 aa, Homo sapiens
LRLMEENWNA

SEQ ID NO: 81 - protein, 10 aa, Homo sapiens
FLDRRGDHEV

SEQ ID NO: 82 - protein, 9 aa, Homo sapiens
CLVDGLTDA

SEQ ID NO: 83 - protein, 9 aa, Homo sapiens
NMIYITCHL

SEQ ID NO: 84 - protein, 10 aa, Homo sapiens
TFLLHDPRPV

SEQ ID NO: 85 - protein, 10 aa, Homo sapiens
KLIRAADLTL

SEQ ID NO: 86 - DNA, 20 bp, synthetic construct
  misc_feature 1..20: note = mouse Zp3 RT-PCR Forward primer
  source 1..20: mol_type = other DNA
gagcttttcggcatttcaag

SEQ ID NO: 87 - DNA, 20 bp, synthetic construct
  misc_feature 1..20: note = mouse Zp3 RT-PCR Reverse primer
  source 1..20: mol_type = other DNA
agcttatcggggatctggtt

SEQ ID NO: 88 - DNA, 23 bp, synthetic construct
  misc_feature 1..23: note = mouse Ppia RT-PCR Forward primer
  source 1..23: mol_type = other DNA
catcctaaagcatacaggtcctg

SEQ ID NO: 89 - DNA, 20 bp, synthetic construct
  misc_feature 1..20: note = mouse Ppia RT-PCR Reverse primer
  source 1..20: mol_type = other DNA
tccatggcttccacaatgtt

SEQ ID NO: 90 - DNA, 20 bp, synthetic construct
  misc_feature 1..20: note = human ZP3 RT-PCR Forward primer
  source 1..20: mol_type = other DNA
atgcaggtaactgacgatgc

SEQ ID NO: 91 - DNA, 20 bp, synthetic construct
  misc_feature 1..20: note = human ZP3 RT-PCR Reverse primer
  source 1..20: mol_type = other DNA
ccatcagacgcagagagaaa

SEQ ID NO: 92 - DNA, 24 bp, synthetic construct
  misc_feature 1..24: note = human FSHR RT-PCR Forward primer
  source 1..24: mol_type = other DNA
tgggctcaggatgtcatcatcgga

SEQ ID NO: 93 - DNA, 24 bp, synthetic construct
  misc_feature 1..24: note = human FSHR RT-PCR Reverse primer
  source 1..24: mol_type = other DNA
tggatgactcgaagcttggtgagg

SEQ ID NO: 94 - DNA, 20 bp, synthetic construct
  misc_feature 1..20: note = human LHR RT-PCR Forward primer
  source 1..20: mol_type = other DNA
ctgagtggctgggactatga

SEQ ID NO: 95 - DNA, 20 bp, synthetic construct
  misc_feature 1..20: note = human LHR RT-PCR Reverse primer
  source 1..20: mol_type = other DNA
ccaaatcaggaccctaagga

SEQ ID NO: 96 - DNA, 22 bp, synthetic construct
  misc_feature 1..22: note = human PPIA RT-PCR Forward primer
  source 1..22: mol_type = other DNA
gccaagactgagtggttggatg

SEQ ID NO: 97 - DNA, 23 bp, synthetic construct
  misc_feature 1..23: note = human PPIA RT-PCR Reverse primer
  source 1..23: mol_type = other DNA
gagttgtccacagtcagcaatgg

SEQ ID NO: 98 - DNA, 20 bp, synthetic construct
  misc_feature 1..20: note = mouse Zp3 qPCR Forward primer
  source 1..20: mol_type = other DNA
ccaacgaccagactgtggaa

SEQ ID NO: 99 - DNA, 20 bp, synthetic construct
  misc_feature 1..20: note = mouse Zp3 qPCR Reverse primer
  source 1..20: mol_type = other DNA
aggactatagctgccagggt

SEQ ID NO: 100 - DNA, 23 bp, synthetic construct
  misc_feature 1..23: note = mouse Ppia qPCR Forward primer
  source 1..23: mol_type = other DNA
catacaggtcctggcatcttgtc

SEQ ID NO: 101 - DNA, 22 bp, synthetic construct
  misc_feature 1..22: note = mouse Ppia qPCR Reverse primer
  source 1..22: mol_type = other DNA
agaccacatgcttgccatccag

SEQ ID NO: 102 - DNA, 19 bp, synthetic construct
  misc_feature 1..19: note = human ZP3 qPCR Forward primer
  source 1..19: mol_type = other DNA
tggcaacagcatgcaggta

SEQ ID NO: 103 - DNA, 20 bp, synthetic construct
  misc_feature 1..20: note = human ZP3 qPCR Reverse primer
  source 1..20: mol_type = other DNA
ctgagtggctgggactatga

SEQ ID NO: 104 - DNA, 20 bp, synthetic construct
  misc_feature 1..20: note = human FSHR qPCR Forward primer
  source 1..20: mol_type = other DNA
gccaagagagcaaggtgaca

SEQ ID NO: 105 - DNA, 20 bp, synthetic construct
  misc_feature 1..20: note = human FSHR qPCR Reverse primer
  source 1..20: mol_type = other DNA
ctcgaagcttggtgaggaca

SEQ ID NO: 106 - DNA, 22 bp, synthetic construct
  misc_feature 1..22: note = human LHR qPCR Forward primer
  source 1..22: mol_type = other DNA
ccggtctcactcgactatcact

SEQ ID NO: 107 - DNA, 23 bp, synthetic construct
  misc_feature 1..23: note = human LHR qPCR Reverse primer
  source 1..23: mol_type = other DNA
aagcttgagatgggatcactttg

SEQ ID NO: 108 - DNA, 20 bp, synthetic construct
  misc_feature 1..20: note = human PPIA qPCR Forward primer
  source 1..20: mol_type = other DNA
gttcttcgacattgccgtcg

SEQ ID NO: 109 - DNA, 21 bp, synthetic construct
  misc_feature 1..21: note = human PPIA qPCR Reverse primer
  source 1..21: mol_type = other DNA
tgtctgcaaacagctcaaagg

(19) United States
(12) Patent Application Publication
(10) Pub. No.: US 2025/0026818 A1
(43) Pub. Date: Jan. 23, 2025

(54) MALE CONTRACEPTION

(71) Applicant: Sciencemed Spóka z ograniczona odpowiedzialnoci, Poznan (PL)

(72) Inventors: Slawomir Wolczynski, Bialystok (PL); Leszek Antoni Pawelczyk, Drwesa (PL); Nafis Ahmed Rahman, Turku (FI)

(73) Assignee: Sciencemed Spóka z ograniczon odpowiedzialnoci, Poznan (PL)

(21) Appl. No.: 18/701,653

(22) PCT Filed: Oct. 18, 2022
(86) PCT No.: PCT/EP2022/078951

§ 371 Date: Apr. 16, 2024

(30) Foreign Application Priority Data
Oct. 19, 2021 (EP) 21203510.9

(51) Int. Cl. C07K 16/18; A61K 35/17; A61K 45/06

(52) U.S. Cl. CPC C07K 16/18; A61K 35/17; A61K 45/06

(57) ABSTRACT

The present invention, generally speaking, relates to methods of contraception in male subjects, which may be a human or a non-human mammal. The present inventors have surprisingly found that the zona pellucida (ZP) proteins, especially the zona pellucida 3 protein (ZP3), is expressed in spermatogonia, spermatocytes and round and elongated spermatids of human and mouse testis, but absent in mature spermatozoa, Sertoli, Leydig, spermatogonial stem and progenitor cells. Based on these findings, zona pellucida proteins, in particular ZP3, constitute suitable targets for immunocontraceptive strategies, especially strategies aimed at inducing transient and reversible male infertility.

### FIELD OF THE INVENTION

The present invention, generally speaking, relates to methods of contraception in male subjects, which may be a human or a non-human mammal. More specifically, the invention relates to antigen sources providing at least an immunogenic portion of a protein expressed by spermatogonia, spermatocytes and spermatids but not by testicular Sertoli, Leydig, spermatogonial stem and progenitor cells, which preferably is a zona pellucida (ZP) protein, especially a ZP3 protein. Such antigen sources may be used in vaccines and pharmaceutical compositions for reversible male contraceptive treatments.

### BACKGROUND OF THE INVENTION

Women have a variety of contraceptive methods to choose from, but male contraceptive choices are still limited. Condoms are the most popular option, suffer from drawbacks, such a decrease in sensations and the fact that their use requires diligence. A second option is vasectomy, but this method is invasive and largely irreversible. Hormone-based (androgen alone or in combination with a progestin) male contraceptive methods have been tested extensively, but have failed to suppress spermatogenesis in all men completely. Moreover, these methods involve the administration of relatively high doses of testosterone and progestin preparations, which often cause side effects, such as acne, mood changes, weight gain, night sweats and altered libido. There is lingering concern about the safety of long-term use of androgens. Currently, several projects (such as, combined nestorone-testosterone gel or male contraceptive pill using a progestogenic androgen-dimethandrolone 17 beta-undecatone) to develop a hormone based male contraceptive are ongoing, but adequate efficacy and safety have not been proven yet.

It is clear that development of a male contraceptive is a major medical challenge and there is still a need for an effective reversible male contraceptive. The ideal male contraceptive will be effective, safe, fully reversible, and accessible to a broad population of potential users. Other considerations are reducing the time taken for a regimen to be effective and reversed, minimizing and identifying “non-responders” and determining long-term safety.

It is an object of the present invention to provide novel immunotherapeutic strategies for male contraception.

### SUMMARY OF THE INVENTION

The present inventors have surprisingly found that the zona pellucida (ZP) proteins, especially the zona pellucida 3 protein (ZP3), is expressed in spermatogonia, spermatocytes and round and elongated spermatids of human and mouse testis, but absent in mature spermatozoa, Sertoli, Leydig, spermatogonial stem and progenitor cells. Based on these findings, zona pellucida proteins, in particular ZP3, constitute suitable targets for immunocontraceptive strategies, especially strategies aimed at inducing transient and reversible male infertility.

ZP3 is normally found in females in the so-called ‘zona pellucida’ that forms an extracellular matrix surrounding the oocyte. This zona pellucida induces acrosome reaction on sperm, determines the species specificity for fertilization and prevents polyspermy in mammals.

Immunocontraception in females based on ZP proteins is well-established, in particular in large wild animals (see e.g. Gupta and Minhas, Frontiers In Bioscience, 2017, Scholar, 9, 357-374, http://www.bioscience.org/2017/v9s/af/492/2.htm; Mask et al., 2015, Theriogenology; 84(2):261-267). Numerous studies have been conducted, wherein reduction in fertility was associated with antibody titers (Shresta et al., 2015, Vaccine 33, 133-140; Harris et al., 1999, Protein Expr Purif; 16(2):298-307; Martinez and Harris, 2000, J Reprod Fertil.; 120(1):19-32).

WO89/03399 concerns immunocontraception in females using monoclonal Zona Pellicuda antibodies or Zona Pellucida anitgens (eliciting antibody response). According to WO 89/03399, anti-ZP antibody inhibits fertilization by interfering with sperm binding to or penetration of the zona pellucida without having adverse effects on ovarian function. It is stated that the method can be designed to induce transient infertility by affecting the sperm zona pellucida reaction.

The present method does not rely on inhibiting sperm binding to or penetration of the zona pellucida, but rather on the inhibition of spermatogenesis in the testis, i.e by eliciting an immune response against the spermatogonia, spermatocytes and/or spermatids that have been found to express ZP protein.

The concept of by eliciting humoral and/or cellular immune responses against ZP proteins and ectopically ZP expressing cancer cells has been proposed and demonstrated for the treatment of certain cancer types. Rahman et al. (A novel treatment strategy for ovarian cancer based on immunization with zona pellucida protein (ZP) 3. FASEB J. 2012 January; 26(1):324-33) demonstrate that malignant ovarian tumors in transgenic mice by eliciting humoral and cellular vaccination against ZP3 proteins and ZP3 expressing cells, respectively. These and similar findings have also been described in international patent publications WO2007/058536, which is directed to immunotherapeutic methods of treating ovarian cancer; WO2019/086507, which is directed to immunotherapeutic methods of treating lung cancer; WO2012/026820, which is directed to immunotherapeutic methods of treating prostate cancer; and WO2016080830, which is directed to immunotherapeutic methods of treating pancreatic cancer

Expression of ZP protein in human or animal spermatogonia, spermatocytes and spermatids, or any other type of cell normally found in testis, has never been established before. The present invention therefore provides for the first time, methods of contraception in male subjects, based on humoral and/or cellular immune responses against ZP proteins. Owing to the fact that testicular Sertoli, Leydig, spermatogonial stem and progenitor cells do not express ZP protein (and hence will not be targeted by the immune response), spermatogenesis may restart and normal fertility may be regained after discontinuation of the ZP3 treatment and waning of the immune response. The fact that the immune response against ZP proteins could be transient has been shown by Rahman et al. (FASEB J. 2012 January; 26(1):324-33, as referenced here above). These findings imply that the state of infertility induced by the methods of the invention could be transient/reversible.

Hence, a first aspect of the invention concerns a method of treatment of a male subject, in particular a method for contraception in a male subject, said method comprising the administration of a pharmaceutical composition comprising:
a) a source of an immunogenic polypeptide comprising at least one of a class I MHC- and a class II MHC-restricted epitope from the amino acid sequence of a human Zona Pellucida protein (hZP);
b) a T cell comprising a T cell receptor that binds an MHC-peptide complex, wherein the peptide is a peptide from the amino acid sequence of an hZP; or,
c) an antibody or fragment thereof that specifically binds to hZP;
wherein preferably, the hZP protein is a human Zona Pellucida 3 protein (hZP3).

A further aspect of the invention concerns a pharmaceutical composition comprising:
a) a source of an immunogenic polypeptide comprising at least one of a class I MHC- and a class II MHC-restricted epitope from the amino acid sequence of a human Zona Pellucida protein (hZP);
b) a T cell comprising a T cell receptor that binds an MHC-peptide complex, wherein the peptide is a peptide from the amino acid sequence of an hZP; or,
c) an antibody or fragment thereof that specifically binds to hZP;
wherein preferably, the hZP protein is a human Zona Pellucida 3 protein (hZP3), for use in a method of treatment of a male subject, in particular a method for contraception in a male subject.

Yet, a further aspect of the invention concerns the use of:
a) a source of an immunogenic polypeptide comprising at least one of a class I MHC- and a class II MHC-restricted epitope from the amino acid sequence of a human Zona Pellucida protein (hZP);
b) a T cell comprising a T cell receptor that binds an MHC-peptide complex, wherein the peptide is a peptide from the amino acid sequence of an hZP; or,
c) an antibody or fragment thereof that specifically binds to hZP;
wherein preferably, the hZP protein is a human Zona Pellucida 3 protein (hZP3), in the manufacture of a pharmaceutical composition for use in method of treatment of a male subject, in particular a method for contraception in a male subject.

Other aspects of the invention concern a pharmaceutical composition, comprising:
a) a source of an immunogenic polypeptide comprising at least one of a class I MHC- and a class II MHC-restricted epitope from the amino acid sequence of a human Zona Pellucida protein (hZP);
b) a T cell comprising a T cell receptor that binds an MHC-peptide complex, wherein the peptide is a peptide from the amino acid sequence of an hZP; or,
c) an antibody or fragment thereof that specifically binds to hZP;
wherein preferably, the hZP protein is a human Zona Pellucida 3 protein (hZP3); and a kit comprising a package containing one or more of such pharmaceutical unit dosage forms as well as a leaflet containing printed instructions to administer one or more of said unit dosage forms in/as a method of contraception in a male subject.

It will be understood that these aspects of the invention all involve the same compositions, the same methods of treatment, the same subjects, etc. unless specifically stated otherwise. Specific details and preferred embodiments of the afore-mentioned methods as well as of the compositions and pharmaceutical kits used therein will become evident to those skilled in the art on the basis of the following detailed description and the appended experimental part.

### DETAILED DESCRIPTION OF THE INVENTION

For the methods of the invention, the subject to be treated is preferably a male and may be a human or a non-human mammal. Preferably, the subject to be treated is a human male. The present methods can be practiced in/on any male subject of reproductive age. In preferred embodiments of the invention the subject is a human male aged 16 years or older, preferably 18 years or older, 20 years or older, 25 years or older, 30 years of older or 35 years or older.

In certain embodiments of the invention, the subject to be treated does not suffer from lung cancer, prostate cancer, or pancreatic cancer and/or any pathology directly associated therewith. In certain embodiments of the invention, the subject to be treated does not suffer from lung cancer, prostate or pancreatic cancer and/or any pathology directly associated therewith, wherein the cancer cells express one or more or all of the ZP proteins. In further embodiments of the invention the subject does not suffer from any disease or pathology involving ZP proteins and/or ZP expressing cells. In certain embodiments of the invention, the subject is a healthy subject.

The terms “treat”, “treating” or “treatment”, as used herein in direct conjunction with the/a ‘subject’ (for example: “method of treating a subject”), typically refers to the act of administering the pharmaceutical composition to said subject, irrespective of whether it is for therapeutic or non-therapeutic purposes. This is not to be confused with the terms “treat”, “treating” or “treatment”, as used in conjunction with a specific disease or symptom (for example: “method of treating disease . . . ”), in which case the terms do imply a therapeutic or prophylactic purpose.

As will be evident from the explanations herein before, the methods of treating male subjects in accordance with the invention, generally speaking, affect spermatogenesis so as to cause a state of infertility, reduced fertility, inability to impregnate female sexual partners, etc. Hence, in a particularly preferred embodiment of the invention, a method as defined herein is provided, wherein said method is a method of contraception in a male. In other embodiments of the invention, a method as defined herein is provided, wherein said method is a method of reducing male fertility, a method of inducing male infertility, a method of inhibiting spermatogenesis, a method of inducing aspermia, a method of inducing azoospermia, a method of reducing significantly sperm count, a method of inducing a state of severe oligospermia, a method of reducing MOT, a method of reducing TMS and/or a method of reducing semen volume. In one embodiment of the invention, the method results in sperm count less than 5·10⁶ sperm/ml of ejaculate, such as less than 2.5·10⁶ sperm/ml of ejaculate, less than 1·10⁶ sperm/ml of ejaculate, less than 5·10⁵ sperm/ml of ejaculate or less than 1·10⁵ sperm/ml of ejaculate, or best simply no sperm.

In preferred embodiments of the present invention, the effects of the treatment, as defined here above, are reversible or transient. In particularly preferred embodiments of the present invention, male fertility parameters, such as sperm count, sperm motility, MOT, TMS and semen volume, return to more than 50% of the baseline level after termination of the treatment, wherein baseline level refers to the level prior to initiation of the treatment, such as more than 60%, more than 70%, more than 80% or more than 90%. In particularly preferred embodiments of the present invention, male fertility parameters, such as sperm count, sperm motility, MOT, TMS and semen volume, return to more than 50% of the average levels for normal fertile males after termination of the treatment, such as more than 60%, more than 70%, more than 80% or more than 90%. Typically, said male fertility parameters return to the levels indicated, within a period of 5 years of termination of the treatment, such as within a period of 4 years, within a period of 3 years, within a period of 2.5 years, within a period of 2 years, within a period of 1.5 years, within a period of 1 year, within a period of 10 months, within a period of 8 months, within a period of 6 months, within a period of 5 months, within a period of 4 months or within a period of 3 months.

As will be understood by those skilled in the art based on the present teachings, the methods of the invention may entail the repeated treatment of the male subject, i.e. by the administration of a booster dose of the pharmaceutical composition as defined herein. Hence, in certain embodiments of the invention, methods as defined herein are provided, wherein the male subject is treated by the administration of a contraceptive effective amount of the pharmaceutical composition once every 3 years, once every 2 years, once every year, once every 10 months, once every 8 months, months, once every 6 months, once every 5 months, once every 4 months, once every 3 months, once every two months or once every month. As will be understood by those skilled in the art based on the present teachings, the treatment of the male subject according to this regimen may be continued for as long as the male subject desires to maintain a state of infertility (or, in case the male subject is a non-human mammal, for as long as it is desired for said non-human mammal to remain infertile). In certain embodiments of the invention, the method comprises the repeated administration of a pharmaceutical composition of the invention, preferably in accordance with the regimens defined herein, for a period of at least 6 month, more preferably at least 9 month, at least 12 months, at least 2 years, at least 3 years, at least 4 years or at least 5 years.

As used herein, the term “contraceptively effective” means adequate for a contraceptive effect”, as will be understood by those skilled in the art, based on the present teachings. Typically, the “contraceptively effective amount” refers to a dose or amount that is adequate to attain any one or any combination of effects defined in the foregoing and/or a dose or amount that is adequate to elicit a primary (auto)immune response directed against (native) ZP glycoproteins and cells expressing ZP proteins. Such effective dosages will depend on a variety of factors including the condition and general state of health of the patient. Thus, dosage regimens can, based on the present teachings, be determined and adjusted by trained medical personnel to provide the optimum therapeutic or prophylactic effect.

The term “pharmaceutical composition” as used herein, refers to any compound, material, compositions and/or dosage form, which is, within the scope of sound medical judgment, suitable for some form of (par)enteral administration to a mammal, especially a human, without causing excessive toxicity, irritation, allergic response and other complications, commensurate with a reasonable benefit/risk ratio.

As defined herein before, the methods of the invention comprise the administration of a pharmaceutical composition comprising:
a) a source of an immunogenic polypeptide comprising at least one of a class I MHC- and a class II MHC-restricted epitope from the amino acid sequence of a human Zona Pellucida protein (hZP);
b) a T cell comprising a T cell receptor that binds an MHC-peptide complex, wherein the peptide is a peptide from the amino acid sequence of an hZP; or,
c) an antibody or fragment thereof that specifically binds to hZP;
wherein preferably, the hZP protein is a human Zona Pellucida 3 protein (hZP3), and wherein the composition is typically administered in a contraceptively effective dose or amount.

The term “Zona Pellucia protein”, as used herein generally refers to any of the proteins found in the human or mammalian zona pellucida. The zona pellucida is a specialized extracellular matrix surrounding the developing oocyte (egg, ovum) within each follicle within the ovary. This matrix is thought to be formed by secretions from the oocyte and the follicle granulosa cells and in human oocytes consists of four types of zona pellucida glycoproteins ZP1, ZP2, ZP3 and ZP4, which have different roles in fertilization. The naming of the ZP glycoprotein components has been somewhat inconsistent over the years, employing several criteria, including apparent molecular weight, protein sequence length and sequence identity comparison, which has resulted in a confused nomenclature. Harris et al. [(1994) DNA seq. 96:829-834] proposed a uniform system of nomenclature in which ZP genes were named in order of length of their encoded protein sequence from longest to shortest. Since, under those criteria the mouse ZP genes fell in the order ZP2, then ZP1 and then ZP3, a new system was introduced wherein ZP2 became ZPA, ZP1 became ZPB and ZP3 became ZPC. Hughes et al [(1999) BBA-Gene Structure and Expression 1447:303-306], amongst others, reported that the true human orthologue of the known mouse ZP1 gene is not ZPB, but that there is a distinct human ZP1 gene. It is now generally accepted that there are four distinct (human) ZP glycoprotein families ZP1, ZP2, ZP3 and ZPB [cf. Lefievre et al (2004) Hum. Reprod. 19:1580-1586]. The ZPB glycoprotein according to this nomenclature is now also referred to as ZP4. This nomenclature is for example applied in the Uniprot/SWISSprot, ensEMBL, BLAST (NCBI), SOURCE, SMART, STRING, PSORT2, CDART, UniGene and SOSUI databases, all implemented in the Bioinformatic Harvester (http://harvester.embl.de).

In accordance with this the terms ZP1, ZP2, ZP3 and ZP4 are employed herein to denote the four ZP glycoprotein families, wherein ZP2, ZP3 and ZP4 correspond to ZPA, ZPC and ZPB, respectively, according to the nomenclature proposed by Harris et al. More in particular, the terms hZP1, hZP2, hZP3 and hZP4 as used herein refer to the proteins having polypeptide backbones listed in SEQ ID NO. 1, SEQ ID NO. 2, SEQ ID NO. 3 and SEQ ID NO. 4, respectively, and allelic variants thereof.

In accordance with the present invention, the ZP protein most preferably is ZP3 protein.

Allelic variants of the ZP sequences that can occur in nature are also encompassed by the respective terms ZP and hZP. Allelic variants include in particular variants resulting from single nucleotide polymorphisms (SNPs). SNPs may fall within coding sequences of genes, non-coding regions of genes, or in the intergenic regions between genes. SNPs within a coding sequence will not necessarily change the amino acid sequence of the protein that is produced. An SNP in which both forms lead to the same polypeptide sequence is termed synonymous (sometimes called a silent mutation)—if a different polypeptide sequence is produced they are nonsynonymous. For a variant to be considered an SNP, it must occur in at least 1% of the population. In the context of the present invention ‘allelic variants’ may also include polypeptide sequence variants resulting from (nonsynonymous) mutations, i.e. polypeptide variants resulting from point mutations, insertions, deletions, etc. occurring in less than 1% of the population.

Thus, in accordance with the present invention the terms hZP1, hZP2, hZP3 and hZP4 include ZP proteins which differ from SEQ ID NO. 1, SEQ ID NO. 2, SEQ ID NO. 3 and SEQ ID NO. 4, respectively, by minor sequence modifications. Such modifications include, but are not limited to: changes in one or a few amino acids, including deletions (e.g., a truncated version of the peptide) insertions and/or substitutions.

An ‘allelic variant’ is herein understood to have at least 90%, preferably at least 95%, more preferably at least 97%, still more preferably at least 98%, still more preferably at least 99%, still more preferably at least 99.5% and most preferably at least 99.9% amino acid sequence identity with any of SEQ ID NO. 1, SEQ ID NO. 2, SEQ ID NO. 3 and SEQ ID NO. 4.

Each of the ZP proteins comprises a signal peptide to direct it into a secretory pathway, a zona domain and a transmembrane domain near the carboxyl terminus followed by a short cytoplasmic tail. In embodiments of the invention that comprises the administration of an antibody or fragment thereof that specifically binds to hZP, the antibodies or fragments thereof are preferably directed against ZP extracellular domain. In embodiments of the invention that concern active immunization a source of an immunogenic polypeptide capable of eliciting a cellular immune response against an hZP, in principle any part of the hZP that comprises a class I MHC- and/or class II MHC-restricted T cell epitope can be used.

The amino acid sequence of hZP3 nascent protein contains a N-terminal signal peptide sequence protein (amino acid positions 1-22 in SEQ ID NO: 3), a conserved “ZP extracellular domain” (amino acid positions 23-350 in SEQ ID NO: 3), and a pro-peptide (amino acid positions 351-424 in SEQ ID NO: 3), consisting of a consensus furin cleavage site (CFCS; amino acid positions 351-352 in SEQ ID NO: 3), a polymerization-blocking external hydrophobic patch (EHP), and a C-terminal transmembrane domain (amino acid positions 353-424 in SEQ ID NO: 3). The hZP3 signal peptide is cleaved off during translation and cleavage at the CFCS separates the mature hZP3 extracellular domain protein (consisting of amino acid positions 23-350 in SEQ ID NO: 3) from the EHP, allowing it to incorporate into nascent ZP filaments. Hence, the amino acid backbone of mature hZP3 extracellular domain has the amino acid sequence consisting of amino acid positions 23-350 in SEQ ID NO: 3 of hZP3. This extracellular domain fragment is denominated herein as hZP3(23-350) (SEQ ID NO: 5).

In a preferred embodiment the present invention relates to a method of treatment by active immunization. The method of active immunization preferably comprises administering a source of an immunogenic polypeptide capable of eliciting a cellular immune response against a human Zona Pellucida protein (hZP), preferably hZP3 or hZP3(23-350); a T cell comprising a T cell receptor that binds an MHC-peptide complex, wherein the peptide is a peptide from an hZP, preferably from hZP3 or hZP3(23-350); an antibody or fragment thereof that specifically binds to an hZP, preferably to hZP3 or hZP3(23-350); and a genetic construct comprising a nucleic acid sequence encoding said polypeptide or antibody, wherein the genetic construct is configured to be delivered and expressed in a human.

The source of the immunogenic polypeptide can be a proteinaceous source, a nucleic acid or a combination thereof. The proteinaceous source can e.g. be a composition comprising one or more peptides, polypeptides or proteins that act as immunogen. Alternatively, the source of the immunogenic polypeptide can be a nucleic acid molecule encoding one or more immunogenic peptides, polypeptides or proteins, which nucleic acid molecule, when administered to the subject to be treated expresses the immunogenic peptides, polypeptides or proteins. The nucleic acid molecule can be a DNA, cDNA RNA, mRNA, a variant thereof, a fragment thereof, or a combination thereof, as e.g. described in WO2014/165291 and WO2013/087083. The source of the immunogenic polypeptide can further be a cell, preferably a live cell, that expresses the immunogenic polypeptide or presents an epitope of the immunogenic polypeptide. Preferably, the cell is a microbial, more preferably a bacterium such as e.g. a live-attenuated Listeria monocytogenes, as e.g. described in WO2015/164121. Alternatively, the cellular source of the immunogenic polypeptide of the invention is an autologous or allogeneic dendritic cell that presents at least one epitope of the immunogenic polypeptide in an HLA molecule on its surfaces.

In a preferred embodiment the immunogenic polypeptide comprises a contiguous amino acid sequence selected from the amino acid sequence of an hZP protein, which contiguous amino acid sequence preferably comprises at least one of a class I MHC- and a class II MHC-restricted T cell epitope. More preferably, the immunogenic polypeptide comprises a contiguous amino acid sequence selected from the amino acid sequence of the hZP3 protein (i.e. SEQ ID NO: 3) which contiguous amino acid sequence preferably comprises at least one of a class I MHC- and a class II MHC-restricted T cell epitope, e.g. selected from Tables 1, ∥ and III (as appended to the description), respectively. More preferably, the contiguous amino acid sequence comprises at least one of a class I MHC- and a class II MHC-restricted T cell epitope with a low percentile rank (see Moutaftsi et al., Nat Biotechnol. 2006 July; 24(7):817-9; and Kotturi et al., J Virol. 2007 May; 81(10):4928-40). A class I MHC-restricted T cell epitope with a low percentile rank preferably is an epitope with a percentile rank that is not higher than 1.00, 0.80, 0.40, 0.30, 0.20, 0.15, 0.10 or 0.05 (see e.g. Tables I and III). A class II MHC-restricted T cell epitope with a low percentile rank preferably is an epitope with a percentile rank that is not higher than 2.50, 2.40, 2.05, 2.00, 1.80, 1.60, 1.40, 1.20, 1.10, 1.00, 0.90, 0.70, 0.60, 0.50, 0.40, 0.20. 0.15, 0.10, 0.05 or 0.02. Preferably, the contiguous amino acid sequence is selected from an amino acid sequence from the group of amino acid sequences from proteolytic hZP3 fragments consisting of the sequence of the N-terminal signal peptide (positions 1-22 in SEQ ID NO: 3), the sequence of the mature extracellular domain (positions 23-350 in SEQ ID NO: 3, i.e. SEQ ID NO: 5), and the sequence of the propeptide (amino acid positions 351-424 in SEQ ID NO: 3). Examples of such contiguous amino acid sequences are given in FIG. 13.

The contiguous amino acid sequence from an hZP(3) protein as comprised within the immunogenic polypeptide, preferably comprises an immunologically active (sequence) fragment of the hZP(3) protein. The term “immunologically active fragments thereof” will generally be understood in the art to refer to a fragment of a hZP(3) protein antigen comprising at least an epitope, which means that the immunogenic polypeptide at least comprises 4, 5, 6, 7 or 8 contiguous amino acids from the sequence of the hZP(3) protein antigen. According to the present invention the fragment comprises at least an MHC class I or MHC class II binding peptide presented by such MHC molecule to the immune system. An ‘immunologically active fragment’ according to this invention comprises at least 7, 8, 9, 10, 11, 12, 13, 14, 15, 16 or 17 contiguous amino acids from the sequence of the ZP protein antigen or homologue or analogue thereof. While the majority of the MHC binding peptides are of a length of 9 amino acids, longer peptides can be accommodated by the bulging of their central portion (Guo et al., 1992, Nature; 360(6402):364-366; Speir et al., 2001, Immunity; 14(1):81-92), resulting in binding peptides of length 8 to 15 (Schumacher et al., 1991, Nature; 350(6320):703-706). Examples of MHC class I binding peptides in the sequence of hZP3 are given in Tables 2 and 5. Peptides binding to class II proteins are not constrained in size (Nelson et al., 1999, Rev Immunogenet; 1(1):47-59; Yassai et al., 2002, J Immunol; 168(3):1281-1285) and can vary from 11 to 30 amino acids long (Rammensee 1995, Immunogenetics; 41(4):178-228) possibly even whole proteins. The binding motif however is about 9 amino acids long. MHC class II can accommodate much longer peptides than MHC class I because the ends of the MHC II binding groove are open, hence an epitope (binding into the groove) may be flanked by additional stretches of amino acids on either end. Examples of MHC class II binding peptides in the sequence of hZP3 are given in Table II (i.e. SEQ IDs NO. 39-65). Still more preferably the fragment comprises both a Cytotoxic T Lymphocyte (CTL) and a Helper T Lymphocyte (HTL) epitope. Most preferably however, the fragment is a peptide that requires processing by an antigen presenting cell, i.e. the fragment has a length of at least about 18 amino acids, which 18 amino acids are not necessarily a contiguous sequence from the hZP(3) protein antigen.

The length of a contiguous amino acid sequence from an hZP(3) protein as comprised within the immunogenic polypeptide or the length of the immunogenic polypeptide itself, therefore preferably is at least 18, 19, 20, 21, 22, 25, 27, 30, 33 or 35 amino acids and preferably no more than 100, 80, 60, 50, 45, 40, 35, 33 or 30 amino acids. Preferably the length of a contiguous amino acid sequence from an hZP(3) protein as comprised within the immunogenic polypeptide, or the length of the immunogenic polypeptide itself, is 19-50 or 19-45, more preferably 25-40 amino acids, even more preferably 25-35 and most preferably 25-30 amino acids. From the view point of manufacturability an immunogenic polypeptide with a length of around 25 amino acids is optimal, while still long enough to contain multiple epitopes and force presentation via Antigen Presenting Cells. Suitable examples of such immunogenic polypeptides comprising contiguous amino acid sequence from the hZP3 protein and each comprising one or more MHC class I and/or MHC class II binding peptide are presented in FIG. 13.

The terms “homologues thereof”, as used herein refer to polypeptides which differ from the naturally occurring polypeptide by minor modifications, but which maintain the basic polypeptide and side chain structure of the naturally occurring form. Such changes include, but are not limited to: changes in one or a few amino acid side chains; changes in one or a few amino acids, including deletions (e.g., a truncated version of the peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. As used herein, a homologue or analogue has either enhanced or substantially similar functionality as the naturally occurring polypeptide. Typically, when optimally aligned, such as by the programs GAP or BESTFIT using default parameters, a naturally occurring polypeptide and a homologue thereof share at least a certain percentage of sequence identity. GAP uses the Needleman and Wunsch global alignment algorithm to align two sequences over their entire length, maximizing the number of matches and minimizes the number of gaps. Generally, the GAP default parameters are used, with a gap creation penalty=8 and gap extension penalty=2. For proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752, USA. Alternatively, percent similarity or identity may be determined by searching against databases such as FASTA, BLAST, etc.

A homologue herein is understood to comprise an immunogenic polypeptide having at least 70%, preferably at least 80%, more preferably at least 90%, still more preferably at least 95%, still more preferably at least 98% and most preferably at least 99% amino acid sequence identity with the naturally occurring ZP polypeptides mentioned above and is still capable of eliciting at least the immune response obtainable thereby. A homologue or analogue may herein comprise substitutions, insertions, deletions, additional N- or C-terminal amino acids, and/or additional chemical moieties, such as carbohydrates, to increase stability, solubility and immunogenicity.

In accordance with the present invention, the immunogenic polypeptide that is administered to the human according to the present method, may be or comprise a protein or glycoprotein, a digest of the protein or glycoprotein and/or fragments thereof, which may be in a purified form or may be comprised within a crude composition, preferably of biological origin, such as lysates, sonicates or fixates of prokaryotic or eukaryotic cell lines. More preferably however, the immunogenic polypeptide is or comprises chemically synthesized (poly)peptides or (poly)peptides that have been produced enzymatically in vitro, which may be in a purified form or may be comprised within a crude composition.

The term “epitope” as used herein refers to a portion of an antigen, typically defined by a short peptide, which is capable of eliciting a cellular or humoral immune response when presented in a physiologically relevant context in vivo. A “T cell epitope” refers to a short peptide or portion thereof that binds to an MHC molecule and is recognized by certain T cells when presented in certain MHC molecules. A T cell epitope is capable of inducing a cell mediated immune response via direct or indirect presentation in heterodimeric membrane MHC molecules. Preferably, in the immunogenic polypeptide at least one MHC class II restricted epitope and at least one MHC class I restricted epitope are present within a contiguous amino sequence from the amino acid sequence of the hZP(3) protein, whereby preferably, the MHC class II restricted epitopes and at least one MHC class I restricted epitopes are selected from Tables II, and I and III, respectively. For the sake of clarity, the peptide of the invention preferably comprises at least one MHC class I presented epitope and preferably also at least one MHC class II presented epitope. Each of these epitopes are presentable and will bind to the corresponding specific MHC molecule present on the cells after having been processed as described herein. Each MHC restricted epitope may therefore also be named an MHC binding and/or presentable epitope. Preferably, a specific proteasomal cleavage site generating the C-terminus of such epitope is present exactly after the epitope's amino acid sequence in order to be liberated from the immunogenic polypeptide and presented on the MHC class I molecule. Length requirements are much less strict for MHC class II presented epitopes, therefore a need for precise enzymatic generation of the class II binding peptide is less absolute. Briefly, MHC molecules preferentially bind particular amino acid residues known as “anchor” residues (K. Falk et al., Nature 351:290-96 (1991)). This characterization permits class I and II MHC binding motifs to be recognized within any known peptide sequence (see e.g. Tables 1, 111 and II).

In the present context, the term “MHC restricted epitope” is synonymous with T cell epitope. The term “class I MHC restricted epitope”, as used herein, refers to peptide sequences recognized by cytotoxic T lymphocytes (also called CD8⁺ cells, TCD8 or CTLs) in association with class I MHC. The term “class II MHC restricted epitope”, as used herein, refers to a peptide recognized by helper T cells (also called CD4⁺ cells, TCD4 or HTLs) in association with class II MHC. A “B cell epitope” is the portion of an antigen that is capable of binding to an antigen binding site of an immunoglobulin and therefore capable of stimulating a humoral response without presentation by an MHC molecule. As explained herein before the polypeptide useful in the present invention, or the nucleic acid encoding said polypeptide, comprises at least one T cell epitope. The use of polypeptides that also comprise a B cell epitope is however not excluded from the present invention. The present immunogenic polypeptides may also include multiple T cell epitopes and, optionally a B cell epitope. When multiple epitopes are present in a peptide, the epitopes may be oriented in tandem or in a nested or overlapping configuration wherein at least one amino acid residue may be shared by two or more epitopes.

The immunogenic polypeptide of the invention preferably includes one or more MHC class I restricted epitopes. As is generally known by the skilled person, an antigen comprising a single MHC restricted epitope will be useful only for treating a (small) subset of patients who express the MHC allele product that is capable of binding that specific peptide. It has been calculated that, in humans, vaccines containing CTL epitopes restricted by HLA-A1, -A2, -A3, -A24 and -B7 would offer coverage to approximately 80% of individuals of most ethnic backgrounds. Therefore, if the present method is used to treat a human, it is particularly preferred that the method comprises the administration of a composition comprising one or more different polypeptides comprising one, more preferably two, most preferably three MHC class I binding native ZP, preferably hZP3, epitopes selected from HLA-A1, HLA-A2, HLA-A3, HLA-A24 and HLA-B7 restricted epitopes; or homologues thereof.

According to another embodiment the immunogenic polypeptide of the invention preferably includes one or more MHC class II restricted epitopes. The most frequently found MHC class II allele products in humans include HLA-DR1, -DR3, -DR4 and -DR7. Accordingly, it is preferred that the method comprises the administration of a composition comprising one or more different polypeptides, said one or more different polypeptides comprising one, more preferably two and most preferably three MHC class II binding native ZP, preferably hZP3, epitopes selected from HLA-DR1, HLA-DR3, HLA-DR4 and HLA-DR7 restricted epitopes; or homologues thereof.

In still another embodiment, the method of the invention comprises the administration of a composition comprising one or more polypeptides, said one or more polypeptides comprising one or more MHC class I restricted epitopes and one or more MCH class II restricted epitopes, as described here above and/or in Tables 2, 5 and 3, respectively; or homologues thereof. Even, more preferably said composition comprises an effective amount of one or more different polypeptides that together include essentially all of the MHC class I and MHC class II binding epitopes comprised in one of the native ZP, preferably hZP3, glycoproteins; or homologues of said one or more polypeptides.

In one embodiment, the present method comprises the administration of a composition comprising one or more different immunogenic polypeptides. Preferably, said one or more different polypeptides together comprise at least 50%, more preferably at least 70%, still more preferably at least 80%, still more preferably at least 90% and most preferably at least 95% of the MHC class I and MHC class II restricted epitopes comprised in a native ZP, preferably hZP3 or hZP3(23-350) or homologues of said one or more polypeptides.

In a preferred embodiment, the present method comprises the administration of a composition comprising one or more immunogenic peptides selected from the peptides presented in FIG. 13, e.g. the immunogenic peptides comprising or consisting of an amino acid sequence of one or more of SEQ ID NO.'s 66-75. The composition can thus comprise one of the immunogenic peptides comprising or consisting of an amino acid sequence selected from SEQ ID NO.'s 66-75. Preferably, however, the composition comprises a combination of at least two of the immunogenic peptides comprising or consisting of an amino acid sequence selected from SEQ ID NO.'s 66-75.

In a further preferred embodiment, the present method comprises the administration of a composition comprising one or more immunogenic peptides comprising or consisting of an epitope selected from the epitopes presented in Table III, i.e. SEQ ID NO.'s 76-85. The composition can thus comprise one of the immunogenic peptides comprising or consisting of an amino acid sequence selected from SEQ ID NO.'s 76-85. Preferably, however, the composition comprises a combination of at least two of the immunogenic peptides comprising or consisting of an amino acid sequence selected from SEQ ID NO.'s 76-85. A particularly preferred composition comprises one or more or all of the immunogenic peptides comprising or consisting of the amino acid sequences of SEQ ID NO.'s 79, 80, 81, 83 and 84.

In a preferred embodiment the present method comprises the administration of a source of an immunogenic polypeptide, which polypeptide comprises at least 50%, more preferably at least 70%, still more preferably at least 80%, still more preferably at least 90% and most preferably at least 95% of the complete amino acid backbone of a native ZP, preferably hZP3 or hZP3(23-350), glycoprotein; or a homologue of said polypeptide.

In a particularly preferred embodiment the present method comprises the administration of a composition comprising a source of an immunogenic polypeptide, which polypeptide comprises 90, 95, 97, 98, 99 or 100% of the complete amino acid backbone of the extracellular domain of a native ZP, preferably hZP3 or hZP3(23-350) or a homologue of said polypeptide. The present immunogenic polypeptides as defined herein before, can be glycosylated. Without wishing to be bound by theory it is hypothesized that by glycosylation of these polypeptides the immunogenicity thereof, at least in as far as they elicit a humoral (B cell response), is increased. Therefore, the immunogenic polypeptide as defined herein before, preferably is glycosylated, having a carbohydrate content varying from 10-80, 15-70 or 20-60 wt. %, based on the total weight of the glycoprotein or glycosylated polypeptide. Preferably, said glycosylated immunogenic polypeptide comprises a glycosylation pattern that is similar to that of the corresponding native zona ZP glycoprotein of a human.

In another particularly preferred embodiment, the method comprises administering a source of an immunogenic polypeptide that is a composition comprising an effective amount of a plurality of different overlapping polypeptide fragments of a native ZP, preferably hZP3 or hZP3(23-350), glycoprotein, which different overlapping polypeptide fragments are between 18-60 amino acids in length, and which together comprise at least 50%, more preferably at least 70%, still more preferably at least 80%, still more preferably at least 90% and most preferably at least 95% of the complete amino acid backbone of said native ZP or of the extracellular domain of said native ZP, preferably hZP3 or hZP3(23-350) or homologues of said polypeptides. More preferably, the different overlapping polypeptide fragments 25-40 amino acids, even more preferably 25-35 and most preferably 25-30 amino acids in length. Typically, the amino acid sequence overlap between the different consecutive 18-60 amino acid polypeptide fragments is at least 7 amino acids, preferably at least 8, more preferably at least 9 and most preferably at least 10 amino acids.

The MHC binding motifs for most common MHC class I and II alleles have been described. These motifs itemize the amino acid residues that serve as MHC binding anchors for specific class I and class II MHC alleles. Sophisticated computer-based algorithms that take into account the MHC binding anchors as well as the amino acids sequence of a peptide are used to predict and quantify the binding affinity of the peptide/MHC interaction. Thus, from the input of the known amino acid sequence of Zona Pellucida proteins, these algorithms list all potential T-cell epitopes, each with its corresponding predictive binding score (see e.g. Tables I, III and II). Commonly known bio-informatics tools for these purposes include e.g. HLA_BIND (Parker et al., 1994, J. Immunol. 152:163), SYFPEITHI (Rammensee et al., 1995, Immunogenetics 41, 178-228; Rammensee et al., Landes Bioscience 1997, International distributor Springer Verlag GmbH & Co. KG, Heidelberg, Germany; http://www.syfpeithi.de/), NetMHC (Buus et al., 2003, Tissue Antigens., 62:378-84; Nielsen et al., 2003, Protein Sci., 12:1007-17; Nielsen et al., 2004, Bioinformatics, 20(9):1388-97; http://www.cbs.dtu.dk/services/NetMHC/), TEPITOPE 2000 (Stumiolo et al., 1999, Nature Biotechnology 17, 555-562; http://www.vaccinome.com/pages/597444/), and the (continuously updated) IEDB analysis resources—T Cell epitope prediction tools: http://tools.iedb.org/main/tcell/.

Alternatively, the skilled artisan will be able to determine HTL and CTL binding epitopes experimentally using standard experimentation (Current Protocols in Immunology, Wiley Interscience 2004). In a preferred embodiment, the method comprises administering a composition comprising an effective amount of a plurality of different polypeptide fragments of between 18-100 amino acids in length, of a native ZP, preferably hZP3 or hZP3(23-350), glycoprotein, wherein each polypeptide fragments comprises one or more of said predicted potential MHC I or MHC II restricted epitopes. Preferably, the amino acid sequence of said predicted potential MHC I or MHC II restricted epitopes in the different polypeptide fragments do not overlap. Preferably, the plurality of different polypeptide fragments collectively comprise at least 50, 70, 80, 90 or 95% of the potential MHC I or MHC II epitopes predicted by one or more of the above-mentioned bio-informatics tools.

In some cases it has been observed that the same peptide may bind to several MHC I or II allele products (see e.g. Table I, III and II). In one embodiment, the use of such ‘promiscuous’ MHC binding peptides in the present method is particularly preferred.

The present method of immunization preferably comprises the administration of a source of immunogenic active polypeptide fragments, said polypeptide fragments being selected from Zona Pellucida protein fragments and/or homologues thereof as defined herein before, said polypeptide fragments comprising CTL and/or HTL epitopes restricted by a variety of HLA molecules and which fragments are between 18 and 45 amino acids in length. Peptides having a length between 18 and 45 amino acids have been observed to provide superior immunogenic properties as is described in WO 02/070006. Peptides may advantageously be chemically synthesized and may optionally be (partially) overlapping and/or may also be ligated to other molecules, peptides or proteins. It may also be advantageous to add to the amino- or carboxy-terminus of the peptide chemical moieties or additional (modified or D-) amino acids in order to increase the stability and/or decrease the biodegradability of the peptide. To improve the immunogenicity/immuno-stimulating properties, moieties may be attached, e.g. by lipidation, elongation and/or conjugation (see below). The peptide can e.g. be elongated by addition of charged or polar amino acids, in order to enhance its solubility and/or increase its stability in vivo.

For immunization purposes the aforementioned immunogenic polypeptides of the invention may also be fused with proteins such as, but not limited to, tetanus toxin/toxoid, diphtheria toxin/toxoid or other carrier molecules. The polypeptides of the invention may also be advantageously fused to heat shock proteins, such as recombinant endogenous (murine) gp96 (GRP94) as a carrier for immunodominant peptides as described in (references: Rapp U K and Kaufmann S H, Int Immunol. 2004 April; 16(4):597-605; Zugel U, Infect Immun. 2001; 69(6):4164-7) or fusion proteins with Hsp70 (Triebel et al; WO99/54464). The immunogenic polypeptides of the invention can also be conjugated with molecules having adjuvant activity as listed herein below, in particular TLR ligands/agonists as listed herein below.

The individual amino acid residues of the present immunogenic (poly)peptides of the invention can be incorporated in the peptide by a peptide bond or peptide bond mimetic. A peptide bond mimetic of the invention includes peptide backbone modifications well known to those skilled in the art. Such modifications include modifications of the amide nitrogen, the α-carbon, amide carbonyl, complete replacement of the amide bond, extensions, deletions or backbone cross-links. See, generally, Spatola, Chemistry and Biochemistry of Amino Acids, Peptides and Proteins, Vol. VII (Weinstein ed., 1983). Several peptide backbone modifications are known, these include, ψ[CH₂S], ψ[CH₂NH], ψ[CSNH₂], ψ[NHCO], ψ[COCH₂] and ψ[(E) or (Z) CH═CH]. The nomenclature used above, follows that suggested by Spatola, above. In this context, .psi. indicates the absence of an amide bond. The structure that replaces the amide group is specified within the brackets.

Amino acid mimetics may also be incorporated in the polypeptides. An “amino acid mimetic” as used herein is a moiety other than a naturally occurring amino acid that conformationally and functionally serves as a substitute for an amino acid in a polypeptide of the present invention. Such a moiety serves as a substitute for an amino acid residue if it does not interfere with the ability of the peptide to elicit an immune response against the native ZP T cell epitopes. Amino acid mimetics may include non-protein amino acids, such as β-, γ-, δ-amino acids, β-, γ-, δ-imino acids (such as piperidine-4-carboxylic acid) as well as many derivatives of L-α-amino acids. A number of suitable amino acid mimetics are known to the skilled artisan, they include cyclohexylalanine, 3-cydohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243-252.

The present method preferably comprises administration of the present immunogenic polypeptides and compositions comprising them via the parenteral or oral route, preferably the parenteral route. Preferred routes of administration include, but are not limited to, intramuscular, intranasal, intraperitoneal, intradermal, subcutaneous, intravenous, intra-arterial, intraocular and oral as well as topically, transdermal, by inhalation or suppository or to mucosal tissue such as by lavage to vaginal, rectal, urethral, buccal and sublingual tissue. Preferred routes of administration include intramuscular, intraperitoneal, intradermal and subcutaneous injection. In another embodiment, administration is into an anatomic site that drains into a lymph node basin. In another embodiment, the administration is into multiple lymph node basins.

Particularly preferred is a combination of an intradermal and a subcutaneous administration of a medicament according to the invention. DC in the epidermis are clearly different from DC in the dermis and in the subcutis. The intracutaneous (intradermal) immunization will cause antigen processing and activation of epidermal DC (Langerin-positive Langerhans cells) that through their dendritic network are in close contact with the keratinocytes. This will also optimally activate inflammatory pathways in the interactions between Langerhans cell and keratinocytes, followed by trafficking of antigen loaded and activated Langerhans cell to the skin-draining lymph nodes. The subcutaneous administration will activate other DC subsets that will also become loaded with antigen and travel independently to the skin-draining lymph nodes. Conceivably, the use of a medicament which may be administered both intradermally and subcutaneously may lead to a synergistic stimulation of T-cells in these draining nodes by the different DC subsets.

Pharmaceutical compositions comprising one or more of the present immunogenic polypeptides as defined herein above, often will also comprise at least one excipient. Excipients are well known in the art of pharmacy and may for instance be found in textbooks such as Remington's pharmaceutical sciences, Mack Publishing, 1995.

According to one preferred embodiment typical dosage regimens comprise administering a dosage of 1-1000 μg per peptide per immunization, more preferably 10-500 μg per peptide per immunization, still more preferably 5-150 μg per peptide per immunization, at least once. Preferably administration of the dosage is repeated one, two, three or more times at intervals of 2, 3 or 4 weeks. According to a preferred embodiment 5-150 μg per peptide per immunization is administered and repeated within 2-3 weeks for one or more times per treatment.

In the present method the one or more immunogenic polypeptides are typically administered at a dosage of about 1, 2, 5, 10, 20, 50, 100, 200, 500 or 1000 μg per immunogenic polypeptide or nucleic acid molecule or more at least once. Preferably administration of the dosage is repeated one, two, three or more times at intervals of 2, 3 or 4 weeks.

In another preferred embodiment, the source of the immunogenic polypeptide of the invention to be administered comprises a nucleic acid molecule encoding an immunogenic polypeptide as defined herein before. Compositions comprising such nucleic acid molecule encoding an immunogenic polypeptide, can comprise one or more different nucleic acid molecules encoding any one of the immunogenic polypeptides, polypeptide fragments, and/or peptides as herein defined above. In addition, the nucleic acid molecule can encode a larger part of a native ZP. The nucleic acid molecule can e.g. encode a polypeptide comprises at least 50, 70, 80, 90, 95 or 100% of the complete amino acid backbone of a ZP, preferably of hZP3, more preferably of hZP3(23-350) or a homologue of said polypeptide. Preferably the nucleic acid molecule encodes a contiguous stretch of at least 50, 70, 80, 90, 95 or 99% of the complete amino acid backbone. It is also possible that a nucleic acid molecule encodes more than one (T cell epitope containing) immunologically active fragments of contiguous amino sequences from an hZP(3) as defined hereinabove, whereby in the encoded amino acids sequences, the different immunologically active fragments can be separated by spacer or linker sequences as beads on a string.

A nucleic acid molecule encoding an immunogenic polypeptide of the invention can be a DNA molecule, preferably a genetic construct wherein the nucleotide sequence coding for the immunogenic polypeptide (cDNA) is operably linked to appropriate expression regulatory sequence that ensure functional expression of the immunogenic polypeptide in the target cells in the human subject, e.g. including at least a strong (e.g. viral) promoter. Genetic constructs for use as DNA vaccines, including e.g. plasmids or viral vectors, are inter alia described in WO2014/165291, WO2016/123285 and WO2017/136758.

Alternatively, a nucleic acid molecule encoding the immunogenic polypeptide of the invention can be an RNA molecule, e.g. an mRNA, ssRNA, dsRNA or combinations thereof. The RNA molecule can e.g. be formulated in a particle comprising the molecule.

In certain particularly preferred embodiments of the present invention, the RNA is messenger RNA (mRNA) that relates to a RNA transcript which encodes an immunogenic polypeptide of the invention. As established in the art, mRNA generally contains a 5-untranslated region (5′-UTR), a peptide coding region and a 3-untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA. In one embodiment, the RNA may have modified nucleosides. In some embodiments, the RNA comprises a modified nucleoside in place of at least one (e.g., every) uridine.

Commonly used delivery methods and carrier molecules for RNA vaccines include: naked (m)RNA; naked (m)RNA with in vivo electroporation; protamine complexed (m)RNA; (m)RNA associated with a positively charged oil-in-water cationic nanoemulsion; (m)RNA dendrimer nanoparticles (e.g. mRNA associated with a chemically modified dendrimer and complexed with polyethylene glycol (PEG)-lipid); (m)RNA protamine liposomes (e.g. protamine-complexed mRNA in a PEG-lipid nanoparticle); (m)RNA associated with a cationic polymer (such as polyethylenimine or ‘PEI’); (m)RNA cationic polymer liposomes (e.g. mRNA associated with a cationic polymer, such as PEI, and a lipid component); (m)RNA polysaccharide particles (e.g. mRNA associated with a polysaccharide, for example chitosan, particle or gel); (m)RNA cationic lipid nanoparticles (e.g. mRNA in a cationic lipid nanoparticle, for example 1,2-dioleoyloxy-3-trimethylammoniumpropane (DOTAP) or dioleoyl-phosphatidylethanolamine (DOPE) lipids); (m)RNA cationic-lipid-cholesterol nanoparticles (e.g. mRNA complexed with cationic lipids and cholesterol); and (m)RNA cationic-lipid-cholesterol-PEG nanoparticles (e.g. mRNA complexed with cationic lipids, cholesterol and PEG-lipid); as described by Pardi et al. (mRNA vaccines—a new era in vaccinology. Nat Rev Drug Discov. 2018 April; 17(4):261-279).

Thus, an aspect of the invention relates to a pharmaceutical composition comprising RNA encoding an immunogenic polypeptide as defined herein before, in any of these forms or carrier-types, and to the use of these pharmaceutical composition in any of the methods as defined herein.

In particularly preferred embodiments of the invention, the RNA is in the form of RNA nanoparticles, comprising RNA encoding an immunogenic polypeptide as defined herein before,

In preferred embodiments of the invention, the RNA nanoparticles have an average diameter that in one embodiment ranges from about 50 nm to about 1000 nm, from about 75 nm to about 800 nm, from about 100 to about 700 nm, from about 125 to about 600 nm, from about 150 nm to about 500 nm, or from about 175 nm to about 400 nm.

In one embodiment, RNA nanoparticles described herein exhibit a polydispersity index less than about 0.5, less than about 0.4, or less than about 0.3. By way of example, the RNA nanoparticles can exhibit a polydispersity index in a range of about 0.1 to about 0.3.

In particularly preferred embodiments of the invention, the RNA nanoparticles are lipid nanoparticles, i.e., particles that contain lipid, in particular cationic lipid, and RNA. Electrostatic interactions between positively charged liposomes and negatively charged RNA results in complexation and spontaneous formation of RNA lipid particles. Positively charged liposomes may be generally synthesized using a cationic lipid, such as DOTMA, and additional lipids, such as DOPE. In one embodiment, a RNA lipid particle is a nanoparticle.

The RNA lipid nanoparticles and compositions comprising RNA lipid nanoparticles described herein are useful for delivery of RNA to a target tissue after parenteral administration, in particular after intravenous administration. The RNA lipid nanoparticles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. In one embodiment, the liposomes and RNA lipid nanoparticles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Choi) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipid nanoparticles comprise 1,2-di-O-octadecenyl-3-trimethylammonium propane (DOTMA) and 1,2-di-(9Z-octadecenoyl)-sn-glycero-3-phosphoethanolamine (DOPE). Liposomes may be used for preparing RNA lipid nanoparticles by mixing the liposomes with RNA.

Spleen targeting RNA lipid nanoparticles are described in WO 2013/143683, herein incorporated by reference. It has been found that RNA lipid nanoparticles having a net negative charge may be used to preferentially target spleen tissue or spleen cells such as antigen-presenting cells, in particular dendritic cells. Accordingly, following administration of the RNA lipid nanoparticles, RNA accumulation and/or RNA expression in the spleen occurs. Thus, RNA lipid nanoparticles of the disclosure may be used for expressing RNA in the spleen. In an embodiment, after administration of the RNA lipid nanoparticles, no or essentially no RNA accumulation and/or RNA expression in the lung and/or liver occurs. In one embodiment, after administration of the RNA lipid nanoparticles, RNA accumulation and/or RNA expression in antigen presenting cells, such as professional antigen presenting cells in the spleen occurs. Thus, RNA lipid nanoparticles of the disclosure may be used for expressing RNA in such antigen presenting cells. In one embodiment, the antigen presenting cells are dendritic cells and/or macrophages. RNA Lipid nanoparticle Diameter

In one embodiment, the lipid solutions, liposomes and RNA lipid nanoparticles described herein include a cationic lipid. As used herein, a “cationic lipid” refers to a lipid having a net positive charge. Cationic lipids bind negatively charged RNA by electrostatic interaction to the lipid matrix. Generally, cationic lipids possess a lipophilic moiety, such as a sterol, an acyl or diacyl chain, and the head group of the lipid typically carries the positive charge. Examples of cationic lipids include, but are not limited to 1,2-di-O-octadecenyl-3-trimethylammonium propane (DOTMA), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 2,3-di(tetradecoxy)propyl-(2-hydroxyethyl)-dimethylazanium (DMRIE), 1,2-dimyristoyl-sn-glycero-3-ethylphosphocholine (DMEPC), I,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA). Preferred are DOTMA, DOTAP, DODAC, and DOSPA. In specific embodiments, the cationic lipid is DOTMA and/or DOTAP.

An additional lipid may be incorporated to adjust the overall positive to negative charge ratio and physical stability of the RNA lipid nanoparticles. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipid nanoparticles include both a cationic lipid and an additional lipid. In an exemplary embodiment, the cationic lipid is DOTMA and the additional lipid is DOPE. Without wishing to be bound by theory, the amount of the at least one cationic lipid compared to the amount of the at least one additional lipid may affect important RNA lipid nanoparticle characteristics, such as charge, particle size, stability, tissue selectivity, and bioactivity of the RNA. Accordingly, in some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

The electric charge of the RNA lipid nanoparticles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)]. The concentration of RNA and the at least one cationic lipid amount can be determined using routine methods by one skilled in the art.

In one embodiment, at physiological pH the charge ratio of positive charges to negative charges in the RNA lipid nanoparticles is from about 1.6:2 to about 1:2, or about 1.6:2 to about 1.1:2. In specific embodiments, the charge ratio of positive charges to negative charges in the RNA lipid nanoparticles at physiological pH is about 1.6:2.0, about 1.5:2.0, about 1.4:2.0, about 1.3:2.0, about 1.2:2.0, about 1.1:2.0, or about 1:2.0.

It has been found that RNA lipid nanoparticles having such charge ratio may be used to preferentially target spleen tissue or spleen cells such as antigen-presenting cells, in particular dendritic cells. Accordingly, in one embodiment, following administration of the RNA lipid nanoparticles, RNA accumulation and/or RNA expression in the spleen occurs. Thus, RNA lipid nanoparticles of the disclosure may be used for expressing RNA in the spleen. In an embodiment, after administration of the RNA lipid nanoparticles, no or essentially no RNA accumulation and/or RNA expression in the lung and/or liver occurs. In one embodiment, after administration of the RNA lipid nanoparticles, RNA accumulation and/or RNA expression in antigen presenting cells, such as professional antigen presenting cells in the spleen occurs. Thus, RNA lipid nanoparticles of the disclosure may be used for expressing RNA in such antigen presenting cells. In one embodiment, the antigen presenting cells are dendritic cells and/or macrophages.

According to the present disclosure, the compositions described herein may comprise salts such as sodium chloride. Without wishing to be bound by theory, sodium chloride functions as an ionic osmolality agent for preconditioning RNA prior to mixing with the at least one cationic lipid. Certain embodiments contemplate alternative organic or inorganic salts to sodium chloride in the present disclosure. Alternative salts include, without limitation, potassium chloride, dipotassium phosphate, monopotassium phosphate, potassium acetate, potassium bicarbonate, potassium sulfate, potassium acetate, disodium phosphate, monosodium phosphate, sodium acetate, sodium bicarbonate, sodium sulfate, sodium acetate, lithium chloride, magnesium chloride, magnesium phosphate, calcium chloride, and sodium salts of ethylenediaminetetraacetic acid (EDTA).

Generally, compositions comprising RNA lipid nanoparticles described herein comprise sodium chloride at a concentration that preferably ranges from 0 mM to about 500 mM, from about 5 mM to about 400 mM, or from about 10 mM to about 300 mM. In one embodiment, compositions comprising RNA lipid nanoparticles comprise an ionic strength corresponding to such sodium chloride concentrations.

Compositions described herein may comprise a stabilizer to avoid substantial loss of the product quality and, in particular, substantial loss of RNA activity during freezing, lyophilization, spray-drying or storage such as storage of the frozen, lyophilized or spray-dried composition.

In an embodiment the stabilizer is a carbohydrate. The term “carbohydrate”, as used herein refers to and encompasses monosaccharides, disaccharides, trisaccharides, oligosaccharides, and polysaccharides.

In embodiments of the disclosure, the stabilizer is mannose, glucose, sucrose or trehalose. According to the present disclosure, the RNA lipid nanoparticle compositions described herein have a stabilizer concentration suitable for the stability of the composition, in particular for the stability of the RNA lipid nanoparticles and for the stability of the RNA.

According to the present disclosure, the RNA lipid nanoparticle compositions described herein have a pH suitable for the stability of the RNA lipid nanoparticles and, in particular, for the stability of the RNA. In one embodiment, the RNA lipid nanoparticle compositions described herein have a pH from about 5.5 to about 7.5.

According to the present disclosure, compositions that include buffer are provided. Without wishing to be bound by theory, the use of buffer maintains the pH of the composition during manufacturing, storage and use of the composition. In certain embodiments of the present disclosure, the buffer may be sodium bicarbonate, monosodium phosphate, disodium phosphate, monopotassium phosphate, dipotassium phosphate, [tris(hydroxymethyl)methylamino]propanesulfonic acid (TAPS), 2-(Bis(2-hydroxyethyl)amino)acetic acid (Bicine), 2-Amino-2-(hydroxymethyl)propane-I,3-diol (Tris), N-(2-Hydroxy-I,I-bis(hydroxymethyl)ethyl)glycine (Tricine), 3-[[I,3-dihydroxy-2-(hydroxymethyl)propan-2-yl]amino]-2-hydroxypropane-I-sulfonic acid (TAPSO), 2-[4-(2-hydroxyethyl)piperazin-1-yl]ethanesulfonic acid (HEPES), 2-[[I,3-dihydroxy-2-(hydroxymethyl)propan-2-yl]amino]ethanesulfonic acid (TES), 1,4-piperazinediethanesulfonic acid (PIPES), dimethylarsinic acid, 2-morpholin-4-ylethanesulfonic acid (MES), 3-morpholino-2-hydroxypropanesulfonic acid (MOPSO), or phosphate buffered saline (PBS). Other suitable buffers may be acetic acid in a salt, citric acid in a salt, boric acid in a salt and phosphoric acid in a salt.

Certain embodiments of the present disclosure contemplate the use of a chelating agent. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), bis(aminoethyl)glycolether-N,N,N′,N′-tetraacetic acid, iminodiacetic acid, citric acid, tartaric acid, fumaric acid, or a salt thereof. In certain embodiments, the chelating agent is EDTA or a salt of EDTA. In an exemplary embodiment, the chelating agent is EDTA disodium dihydrate.

In some embodiments, the EDTA is at a concentration from about 0.05 mM to about 5 mM,

In embodiments, the composition of the present disclosure is a liquid or a solid. Non-limiting examples of a solid include a frozen form or a lyophilized form. In a preferred embodiment, the composition is a liquid.

The RNA described herein, e.g., formulated as RNA lipid nanoparticles, is useful as or for preparing pharmaceutical compositions or medicaments for therapeutic or prophylactic treatments.

The compositions of the present disclosure may be administered in the form of any suitable pharmaceutical composition. A pharmaceutical composition is also known in the art as a pharmaceutical formulation. In the context of the present disclosure, the pharmaceutical composition comprises the RNA described herein, e.g., formulated as RNA lipid nanoparticles.

Routes of administration for nucleic acid vaccines include, but are not limited to, intramuscular, intranasal, intraperitoneal, intradermal, subcutaneous, intravenous, intra-arterial, intraocular and oral as well as topically, transdermal, by inhalation or suppository or to mucosal tissue such as by lavage to vaginal, rectal, urethral, buccal and sublingual tissue. Preferred routes of administration include intramuscular, intraperitoneal, intradermal and subcutaneous injection. Genetic constructs may be administered by means including, but not limited to, electroporation methods and devices, traditional syringes, needleless injection devices, or “micro projectile bombardment gone guns”.

The source of the immunogenic polypeptide of the invention can further be a live cell that expresses and/or presents the immunogenic polypeptide. The cell can be an autologous or allogeneic immune cell, e.g. a dendritic cell derived from the subject to treated, or the cell can be a microbial cell, more preferably a bacterium such as e.g. a live-attenuated Listeria monocytogenes. The expressed immunogenic polypeptide preferably is an immunogenic polypeptide as defined hereinabove. The immunogenic polypeptide can be expressed as part of a fusion protein, wherein preferably the immunogenic polypeptide is fused to a protein that is endogenous to the organism, e.g. an N-terminal fragment of an L. monocytogenes LLO or ActA protein. Suitable embodiments for Listeria-based vaccines are e.g. described in WO2015/164121. A bacterium expressing the immunogenic polypeptide of the invention can be administered orally or parenterally, preferably intravenously.

In another embodiment of the invention, the source of the immunogenic polypeptide of the invention is an autologous or allogeneic dendritic cell (DC) that presents at least one MHC restricted epitope of the immunogenic polypeptide in an HLA molecule on its surfaces. Such dendritic cells can e.g. be prepared ex vivo by contacting and/or loading DCs from the patient's blood, e.g. DCs isolated from mononuclear cells from the patient/subject, with a composition comprising the immunogenic polypeptide of the inventions. The immunogenic polypeptide contacted with mononuclear cells or DCs preferably is an immunogenic polypeptide as defined hereinabove. A pharmaceutical to facilitate harvesting of DC can be used, such as Progenipoietin™ (Monsanto, St. Louis, Mo.) or GM-CSF/IL-4. After pulsing the DCs with peptides and washing to remove unbound peptides, the DCs are reinfused into the patient. In this embodiment, a composition is provided comprising peptide-pulsed DC which present the pulsed peptide epitopes in HLA molecules on their surfaces. Alternatively, instead of using autologous cells derived from the subject, allogenic DCs can be used that are derived from a precursor human dendritic cell line and designed to deliver antigens, such as e.g. described in WO2009/019320, WO2014/090795 and WO2014/006058. Methods of inducing an immune response employing ex vivo peptide-pulsed DC are well known to the skilled person.

Another aspect of the invention relates to a pharmaceutical preparation comprising as the active ingredient the present source of a polypeptide as defined herein before. More particularly the pharmaceutical preparation comprises as the active ingredient one or more of the aforementioned immunogenic polypeptides selected from the group of ZP proteins, homologues thereof and fragments of said ZP proteins and homologues thereof, or, alternatively, a gene therapy vector as defined herein above.

According to a first embodiment a pharmaceutical preparation is provided comprising one or more of the immunogenic polypeptides of the invention. The concentration of said polypeptide in the pharmaceutical composition can vary widely, i.e., from less than about 0.1% by weight, usually being at least about 1% by weight to as much as 20% by weight or more.

The composition preferably at least comprises a pharmaceutically acceptable carrier in addition to the active ingredient. The pharmaceutical carrier can be any compatible, non-toxic substance suitable to deliver the immunogenic polypeptides or gene therapy vectors to the patient. For polypeptides, sterile water, alcohol, fats, waxes, and inert solids may be used as the carrier. Pharmaceutically acceptable adjuvants, buffering agents, dispersing agents, and the like, may also be incorporated into the pharmaceutical compositions.

According to a particularly preferred embodiment, the present pharmaceutical composition comprises an adjuvant, as defined in more detail herein before. Adjuvants for incorporation in the present composition are preferably selected from the group of ligands that are recognised by a Toll-like-receptor (TLR) present on antigen presenting cells, including lipopeptides (see e.g. WO 04/110486), lipopolysaccharides, peptidoglycans, liopteichoic acids, lipoarabinomannans, lipoproteins (from mycoplasma or spirochetes), double-stranded RNA (poly I:C), unmethylated DNA, flagellin, CpG-containing DNA, Pam3cysSK4, and imidazoquinolines, as well derivatives of these ligands having chemical modifications. The skilled person will be able to determine the exact amounts of any one of these adjuvants to be incorporated in the present pharmaceutical preparations in order to render them sufficiently immunogenic. According to another preferred embodiment, the present pharmaceutical preparation may comprise one or more additional ingredients that are used to enhance CTL immunity as explained herein before.

Formulation of the medicaments of the invention, e.g. composition comprising a source of the immunogenic polypeptide, ways of administration and the use of pharmaceutically acceptable excipients are known and customary in the art and for instance described in “Remington: The Science and Practice of Pharmacy” (Ed. Allen, L. V. 22^{nd} edition, 2012, www.pharmpress.com).

The immunogenic polypeptides, nucleic acids encoding them or cells expressing them for use in the present invention can be prepared using recombinant techniques such as described in Ausubel et al., “Current Protocols in Molecular Biology”, Greene Publishing and Wiley-Interscience, New York (1987) and in Sambrook and Russell (2001) “Molecular Cloning: A Laboratory Manual” (3^{rd} edition), Cold Spring Harbor Laboratory, Cold Spring Harbor Laboratory Press, New York; both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis). However, more preferably, immunogenic polypeptides of the invention or nucleic acids encoding them are prepared by chemical synthesis. Chemical synthesis of peptides or nucleic acids is routine practice and various suitable methods are known to the skilled person. Chemical synthesis of peptides or nucleic acids also overcomes the problems associated with recombinant production, which is more difficult to standardize and requires extensive purification and quality control measures.

In a yet a further aspect the inventions pertain to a T cell comprising a T cell receptor that binds an MHC-peptide complex, wherein the peptide preferably is a peptide comprising or consisting of an MHC class I and MHC class II restricted epitopes comprised in a native ZP, preferably hZP3 or hZP3(23-350), or a homologue of said one or more polypeptides. Such a T cell can e.g. be obtained in a method comprising contacting a T-cell with an antigen presenting cell expressing a polynucleotide encoding an immunogenic polypeptide of the invention and/or contacting a T-cell with an antigen presenting cell loaded with an immunogenic polypeptide of the invention; and, optionally, culturing said T-cell. The antigen presenting cell (APC), preferably is a dendritic cell (DC). The T-cell is preferably a CD8⁺ cytotoxic T-cell or a CD4⁺ T-helper cell. Introducing a polynucleotide encoding the immunogenic polypeptide into the APC or DC may be performed using any method known to the person skilled in the art, preferably a polynucleotide according to the invention is introduced into the APC or DC using transfection. Preferably the polynucleotide encoding the immunogenic polypeptide is provided with proper control sequences, or be comprised in a proper expression vector. Contacting a T-cell with an immunogenic polypeptide of the invention can be performed by any method known to the person skilled in the art. Preferably, the immunogenic polypeptide or an epitope comprised in the immunogenic polypeptide is presented to the CD8⁺ cytotoxic T-cell or CD4⁺ T-helper cell by an MHC class I or an MHC class II molecule on the surface of an APC, preferably a DC. The person skilled in the art knows how to load an APC or DC with a peptide. Culturing said T-cell may be performed using any method known by the person skilled in the art. Maintaining a T-cell under conditions to keep the cell alive is herein also to be construed to be culturing. Preferably, the T-cell according to this aspect of the invention is contacted with an immunogenic polypeptide according to the invention as defined in the first aspect of the invention. Ex vivo methods for obtaining and activating antigen-specific T cells are described in more detail e.g. in WO2017/173321. In this aspect the invention also relates to a composition comprising an (activated) T cell according to the invention, as well as to methods of the inventions, comprising administering to the subject a contraceptively effective amount of an (activated) T cell described herein, or produced by a method described herein. In embodiments, the administering comprises administering from about 10⁶ to 10¹², from about 10⁸ to 10¹¹ or from about 109 to 10¹⁰ of the (activated) specific T cells. The T cell or composition therewith is preferably administered via intravenous, intraperitoneal, intradermal, or subcutaneous administration. In another embodiment, the T cell or composition therewith is administered into an anatomic site that drains into a lymph node basin. In another embodiment, the administration is into multiple lymph node basins. In a preferred embodiment the present invention relates to a method of treatment by passive immunization. The method preferably comprises administering an antibody or fragment thereof that specifically binds to an epitope of human Zona Pellucida (hZP) protein, preferably the antibody or fragment thereof specifically binds to an epitope of hZP3 or hZP3(23-350).

An antibody “which binds” an antigen of interest, is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoassay (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labelled target. In this case, specific binding is indicated if the binding of the labelled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{d} for the target (which may be determined as described below) of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope.

A “K_{d}” or “K_{d} value” can be measured by using surface plasmon resonance assays using a BIAcore™-2000 or a BIAcore™-3000 (BIAcore, Inc., Piscataway, N.J.) at 25.degree. C. with immobilized antigen CM5 chips at about10-50 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μg/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of the antibody or Fab (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (K_{d}) is calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stir red cuvette. An “on-rate” or “rate of association” or “association rate” or “kₒₙ” according to this invention can also be determined with the same surface plasmon resonance technique described above using a BIAcore™-2000 or a BIAcore™-3000 (BIAcore, Inc., Piscataway, N.J.) as described above.

The term “antibody” as used herein is meant in a broad sense and refers to any type of immunoglobulin molecule, including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

One embodiment of the invention concerns methods comprising administering to said subject a composition comprising an anti-ZP antibody, preferably an anti-hZP3 or anti-hZP3(23-350) antibody, wherein the antibody induces killing of cells by antibody-dependent cell-mediated cytotoxicity (ADCC), antibody-dependent cellular phagocytosis (ADCP), complement dependent cytotoxicity (CDC) or apoptosis. As is generally understood by those of average skill in the art these antibody effector functions may be mediated by the Fc portion of the antibody, e.g. by binding of an Fc effector domain(s) to an Fc receptor on an immune cell with phagocytic or lytic activity or by binding of an Fc effector domain(s) to components of the complement system. Typically, the effect(s) mediated by the Fc-binding cells or complement components eventually result in inhibition and/or depletion of target cells, i.e. ZP-expressing cells. Human IgG isotypes IgG1, IgG2, IgG3 and IgG4 exhibit differential capacity for effector functions. ADCC may be mediated by IgG1 and IgG3, ADCP may be mediated by IgG1, IgG2, IgG3 and IgG4, and CDC may be mediated by IgG1 and IgG3. In the methods described herein the anti-hZP3 or anti-hZP3(23-350) antibody preferably is an IgG1, IgG2, IgG3 or IgG4 antibody.

In the methods described herein the anti-hZP3 or anti-hZP3(23-350) antibody induces in vitro and/or in vivo killing of cells that express ZP3 protein by antibody-dependent cellular cytotoxicity (ADCC). In the methods described herein the anti-hZP3 or anti-hZP3(23-350) antibody induces in vitro and/or in vivo killing of cells that express ZP protein by complement-dependent cytotoxicity (CDC). In the methods described herein the anti-hZP3 or anti-hZP3(23-350) antibody induces in vitro and/or in vivo killing of cells that express ZP3 protein by antibody-dependent cellular phagocytosis (ADCP).

In the methods described herein the anti-hZP3 or anti-hZP3(23-350) antibody induces in vitro and/or in vivo killing of cells that express ZP3 protein by apoptosis.

In the methods described herein the anti-ZP antibody can bind human ZP with a range of affinities (K_{D}). In one embodiment according to the invention the anti-ZP antibody binds to ZP with high affinity, for example, with a K_{D} equal to or less than about 10⁻⁷ M, such as but not limited to, 1-9.9 (or any range or value therein, such as 1, 2, 3, 4, 5, 6, 7, 8, or 9)·10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art. One exemplary affinity is equal to or less than 1.10⁻⁸ M. Another exemplary affinity is equal to or less than 1·10⁻⁹ M.

Preferred antibodies for use in the present invention are monoclonal antibodies. Suitable (monoclonal) antibodies can be generated, screened and produced by methods that are well known in the art and e.g. described in textbooks like “Antibodies: A Laboratory Manual, Second edition, Edited by E. A. Greenfield, 2014, Cold Spring Harbor Laboratory Press. More specifically, Ranking et al. (1998, Development 125, 2415-2424) describe the generation of a monoclonal antibody H3.1 against a C-terminal peptide of the hZP3 extracellular domain comprising amino acids 335-350 of SEQ ID NO: 3. More preferably, antibodies for use in the present invention are humanized, or even more preferably human monoclonal antibodies, as may be obtained by methods well known in the art, such as e.g. described resp. in Olimpieri et al. (Bioinformatics. 2015; 31(3): 434-435) and Sheehan and Marasco (Microbiol Spectr. 2015; 3(1):AID-0028-2014) and reference cited therein.

Any antibodies that bind to an extracellular domain of a ZP protein, such as the extracellular domain of hZP3. i.e. hZP3(23-350), which extracellular protein has the amino acid sequence of SEQ ID NO: 5, can be used for the present invention. Examples of suitable antibodies that can be used for the present invention include e.g. antibodies that have the ability to cross-block the binding of one or more of the reference antibodies that are known to bind to hZP3(23-350). One such reference antibody that is known to specifically bind to hZP3(23-350) is the H3.1 antibody described by Ranking et al. (1998, supra), for which the hybridoma is obtainable from ATCC under accession no.: ATCC CRL-2569.

Thus, a preferred antibody for use in the present invention has the ability to cross-block the binding of at least one antibody that is known to specifically bind to a ZP, preferably to hZP3, more preferably to hZP3(23-350) or a homologue of said polypeptide. More preferably, the antibody has the ability to cross-block the binding of the H3.1 antibody with accession no.: ATCC CRL-2569. The ability of an anti-ZP antibody to cross-block the binding of a reference antibody is herein defined as the ability to reduce the binding of the reference antibody to a suitable target molecule comprising a ZP, hZP3 or hZP3(23-350) amino acid sequences by at least 10, 20, 50, 75, 90. 95, 99, 99.9 or 99.99% when the target molecule has first been bound by the anti-ZP antibody, or vice versa (i.e. binding of the anti-ZP antibody is reduced when the target molecule is first bound by the reference antibody). The ability to cross-block may in principle be determined using any type of immunoassay, preferably a competitive immunoassay, including e.g. ELISA, solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et 20 al., J. Immunol. 137:3614-3619 (1986)); solid phase direct labeled assay, solid phase direct labeled sandwich assay (see “Antibodies, A Laboratory Manual,” Second edition, 2014; supra); solid phase direct label RIA using I¹²⁵ label (see Morel et al., Molec. Immunol. 25(1):7-15 (1988)); solid phase direct biotinavidin EIA (Cheung et al., Virology 176:546-552 (1990)); and direct labeled RIA (Moldenhauer et al., Scand. J. Immunol. 32:77-82 (1990)). Typically, such an assay involves the use of a purified target molecule bound to a solid surface, an unlabeled test antibody and a labelled reference antibody. Competitive inhibition is measured by determining the amount of label bound to the solid surface in the presence of the test antigen-binding protein. Usually the test antibody is present in excess.

In the methods of the invention described herein the anti-ZP antibody may be provided in suitable pharmaceutical compositions comprising the anti-ZP antibody and a pharmaceutically acceptable carrier. Suitable vehicles and formulations, inclusive of other human proteins, e.g., human serum albumin, are described, for example, in e.g. Remington: The Science and Practice of Pharmacy, 21st Edition, Troy, D. B. ed., Lipincott Wlliams and Wilkins, Philadelphia, Pa. 2006, Part 5, Pharmaceutical Manufacturing pp 691-1092, see especially pp. 958-989.

The mode of administration of the anti-ZP antibody in the methods of the invention described herein may be any suitable route such as parenteral administration, e.g., intradermal, intramuscular, intraperitoneal, intravenous or subcutaneous, pulmonary, transmucosal (oral, intranasal, intravaginal or rectal) or other means appreciated by the skilled artisan, as well known in the art. The anti-ZP antibody in the methods of the invention described herein may be administered to a patient by any suitable route, for example parentally by intravenous (i.v.) infusion or bolus injection, intramuscularly or subcutaneously or intraperitoneally.

In the methods of the invention, the anti-ZP antibody is administered in a contraceptively effective amount and may be sometimes 0.005 mg to about 100 mg/kg, e.g. about 0.05 mg to about 30 mg/kg or about 5 mg to about 25 mg/kg, or about 4 mg/kg, about 8 mg/kg, about 16 mg/kg or about 24 mg/kg, or for example about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

The present invention also relates to a method of contraception in a male subject by targeted therapy, typically by administering a composition comprising an antibody or fragment thereof capable of immunospecifically binding to an epitope of human Zona Pellucida (hZP) protein, preferably an antibody or fragment thereof capable of immunospecifically binding to an epitope of human Zona Pellucida 3 (hZP3) protein, wherein the antibody or fragment is part of an immunoconjugate, such as an immunotoxin or an antibody-drug conjugate.

The term “immunoconjugate” refers to conjugates in which an antibody or fragment thereof is chemically linked to another molecule. When the molecule linked to the antibody or fragment thereof is a toxin, the immunoconjugate is known as an immunotoxin. The use of immunoconjugates may allow targeted delivery of the drug moiety to the spermatogonia, spermatocytes and/or spermatozoa. Maximal efficacy with minimal system exposure is sought thereby. Efforts to design and refine antibody conjugates have focused on the selectivity of monoclonal antibodies (mAbs) as well as drug-linking and drug-releasing properties.

As will be understood by those skilled in the art, the anti-ZP antibody portion can be an anti-ZP antibody having any or all of the characteristics described herein before in relation to the passive immunization embodiment. Immunoconjugates may also comprise an antibody or fragment thereof that is merely capable of immunospecifically binding to an epitope of human Zona Pellucida (hZP) protein, preferably an antibody or fragment thereof capable of immunospecifically binding to an epitope of human Zona Pellucida 3 (hZP3) protein, without inducing any effector mechanisms.

The present methods for immunization may further comprise the administration, preferably the co-administration, of at least one adjuvant. Adjuvants may comprise any adjuvant known in the art of vaccination and may be selected using textbooks like Current Protocols in Immunology, Wiley Interscience, 2004.

Adjuvants are herein intended to include any substance or compound that, when used in combination with an antigen to immunize a human or an animal, stimulates the immune system, thereby provoking, enhancing or facilitating the immune response against the antigen, preferably without generating a specific immune response to the adjuvant itself. Preferred adjuvants enhance the immune response against a given antigen by at least a factor of 1.5, 2, 2.5, 5, 10 or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art. The adjuvant preferably is capable of enhancing the immune response against at least two different antigens. The adjuvant of the invention will usually be a compound that is foreign to a human, thereby excluding immunostimulatory compounds that are endogenous to humans, such as e.g. interleukins, interferons and other hormones.

A number of adjuvants are well known to one skilled in the art. Suitable adjuvants include e.g. Granulocyte-macrophage colony-stimulating factor (GM-CSF), incomplete Freund's adjuvant (IFA), Montanide™ ISA-51, Montanide™ ISA 720 (adjuvants produced by Seppic, France), alpha-galactosylceramide, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxy-phosphoryloxy)-ethylamine (CGP 19835A, referred to as MTP-PE), DDA (2 dimethyldioctadecylammonium bromide), polylC, Poly-A-poly-U, RIBI™, GERBU™, Pam3™, Carbopol™, Specol™, Titermax™, tetanus toxoid, diphtheria toxoid, meningococcal outer membrane proteins, diphtheria protein CRM197. Preferred adjuvants comprise a ligand that is recognised by a Toll-like-receptor (TLR) present on antigen presenting cells. Various ligands recognised by TLR's are known in the art and include e.g. lipopeptides (see e.g. WO 04/110486), lipopolysaccharides, peptidoglycans, liopteichoic acids, lipoarabinomannans, lipoproteins (from mycoplasma or spirochetes), double-stranded RNA (poly I:C), poly ICLC (Hiltonol™, produced by Oncovir, Inc., USA), unmethylated DNA, flagellin, CpG-containing oligonudeotides, growth factors and cyctokines, such as monokines, lymphokines, interleukins, chemokines (e.g. IL-1, IL-2, IL-3, IL-4, IL-5, IL-6, IL-7, IL-8, IL-9, IL-10, IL-12, INFa, INF-y, GM-CSF, LT-a.), Pam3CysSK4 and imidazoquinolines, as well derivatives of these ligands having chemical modifications. The use of poly I:C is particularly preferred.

The method of the invention can suitably be combined with an immunomodulating therapy. It is particularly preferred that when the method of the invention comprises the administration of a source of an immunogenic polypeptide capable of eliciting a cellular or humoral immune response against hZP that such method is combined with an immunomodulating therapy. Immunomodulating therapies that can be combined with the method of the invention can be selected from one or more of: using a checkpoint inhibitor, such as e.g. an antibody against PD1, PDL1, CTLA4, TIM-3 and/or LAG-3; using an antibody targeting selected TNF receptor family members, such as e.g. an antibody against CD40, 4-1 BB, CD137, OX-40/CD134 and/or CD27; using an immunosuppressive cytokine such as e.g. IL-10, TGF-β and/or IL-6; using a cytokine such as e.g. IL-7, IL-15, and IL-21 and/r IL-2, as these cytokines have the capacity to expand antigen-experienced T cells; using a TLR agonist and/or a TLR ligand; using an agonist of invariant natural killer T (iNKT) cells, such as e.g. a synthetic iNKT agonist.

Another aspect of the invention concerns a pharmaceutical composition, comprising:
a) a source of an immunogenic polypeptide comprising at least one of a class I MHC- and a class II MHC-restricted epitope from the amino acid sequence of a human Zona Pellucida protein (hZP);
b) a T cell comprising a T cell receptor that binds an MHC-peptide complex, wherein the peptide is a peptide from the amino acid sequence of an hZP; or,
c) an antibody or fragment thereof that specifically binds to hZP;
as described and defined herein before, wherein preferably, the hZP protein is a human Zona Pellucida 3 protein (hZP3);
typically in combination with one or more pharmaceutically acceptable excipients.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants. The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents, and/or excipients. Suitable preservatives for use in the pharmaceutical compositions of the present disclosure include, without limitation, benzalkonium chloride, chlorobutanol, paraben, and thimerosal. The term “diluent” relates a diluting and/or thinning agent. Moreover, the term “diluent” includes any one or more of fluid, liquid, or solid suspension and/or mixing media. Examples of suitable diluents include ethanol, glycerol, and water. The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier includes, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline. Pharmaceutically acceptable carriers, excipients, or diluents for therapeutic use are well known in the pharmaceutical art, and are described, for example, in Remington's Pharmaceutical Sciences, Mack Publishing Co. (A. R Gennaro edit. 1985). Pharmaceutical carriers, excipients or diluents can be selected with regard to the intended route of administration and standard pharmaceutical practice. The pharmaceutical compositions of the present disclosure preferably comprise one or more adjuvants as defined herein before.
and a kit comprising a package containing one or more of such pharmaceutical unit dosage forms as well as a leaflet containing printed instructions to administer one or more of said unit dosage forms in/as a method of contraception in a male subject.

A further aspect of the present invention provides a pharmaceutical kit comprising a package containing a pharmaceutical composition as defined herein and a leaflet containing printed instructions to administer said pharmaceutical composition to a male subject for contraception in accordance with the methods disclosed herein.

In accordance with embodiments of the invention, the pharmaceutical kit comprises a container, such as a cardboard box, holding a vial containing the composition of the invention and a leaflet inserted into the container, typically a patient information leaflet containing printed information, which information may include a description of the form and composition of the pharmaceutical composition contained in the kit, an indication of the therapeutic indications for which the product is intended, instructions as to how the product is to be used and information and warnings concerning adverse effects and contraindications associated with the use. It will be understood by those of average skill in the art, based on the information presented herein, that the leaflet that is part of the kit according to the invention, will typically contain the information concerning the therapeutic indications, uses, treatment regimens, etc. as described here above in relation to the methods of treatment of the present invention.

Unless otherwise defined, all terms used in disclosing the invention, including technical and scientific terms, have the meaning as commonly understood by one of ordinary skill in the art to which this invention belongs.

By means of further guidance, term definitions are included to better appreciate the teaching of the present invention.

“A”, “an”, and “the” as used herein refer to both singular and plural forms unless the context clearly dictates otherwise. By way of example, “a compartment” refers to one or more than one compartment.

“About” as used herein referring to a measurable value such as a parameter, an amount, a temporal duration, and the like, is meant to encompass variations of +/−10% or less, more preferably +/−5% or less, even more preferably +/−1% or less of and from the specified value, in so far such variations are appropriate to perform in the disclosed invention. However, it is to be understood that the value to which the modifier “about” refers is itself also specifically disclosed.

“Comprise”, “comprising”, “comprises” and “comprised of” as used herein are synonymous with “include”, “including”, “includes” or “contain”, “containing”, “contains” and are inclusive or open-ended terms that specify the presence of what follows, e.g. a component, and do not exclude or preclude the presence of additional, non-recited components, features, element, members, steps, known in the art or disclosed therein.

The recitation of numerical ranges by endpoints includes all numbers and fractions subsumed within that range, as well as the recited endpoints. The skilled person will appreciate that the present invention can incorporate any number of the specific features described above.

### EXAMPLES

### Example 1: Novel Expression of Zona Pellucida 3 Protein in Normal Testis

The expression in the zona pellucida glycoprotein 3 (ZP3), originally thought to be specific for oocytes, was recently extended to ovarian, prostate, colorectal and lung cancers. Earlier successful ZP3 immunization of a transgenic mouse model carrying a ZP3 positive ovarian tumor emphasized the suitability of ZP3 for cancer immunotherapy. This study was carried out to determine whether any other normal tissue besides the ovary in healthy human and mouse tissues may express ZP3, considered important to exclude off-target effects of ZP3 cancer immunotherapy.

### Materials and Methods

### Human Tissue Samples

Normal human tissue samples (n=94; subjects age 30-45 years) were examined from archival paraffin blocks or frozen tissues from the Medical University of Bialystok Department of Pathomorphology and the University Biobank, in Poland (n=10/ovary; n=8/testis; n=7/myometrium smooth muscle; n=7/skeletal muscle; n=6/prostate and epididymis and n=5/small intestine, large intestine, liver, pancreas, stomach, lymph node, brain, lung, kidney and breast for each tissue type). Samples were taken during the surgery and checked by the pathologist to ensure that they were healthy tissues and devoid of any cancer cells. The human testes samples removed during the surgery (n=6), were received from the Medical University of Bialystok Biobank. None of the male patients had any case of infertility or spermatogenic maturation arrest. Testicular structure and spermatogonia, spermatocytes and spermatids were identified and distinguished based on the following characteristics: 1) typical size of the nuclear structure; 2) position in the epithelium; 3) typical pachytene morphology; 4) chromatin condensation patterns. Human semen samples (n=6) were obtained from male patients treated for couple infertility problems (diagnosed female factors) recruited from the IVF program at the Department of Reproduction and Gynecological Endocrinology of the Medical University of Bialystok, Poland. Semen samples were obtained by masturbation after 3 days of sexual abstinence. After liquefaction at room temperature for 30-60 min, semen parameters were assessed according to the WHO guidelines (WHO 2010) and samples were used for the IVF procedure. The remaining samples were used to collect spermatozoa using the swim-up method. Samples with less than 15×10⁶ spermatozoa/ml were excluded from this study. Written informed consent was obtained from all patients before inclusion. The local Human Investigations Ethics Committee at the Medical University of Bialystok, Bialystok Poland approved the study.

### Mouse Tissues Samples

A total of n=40 normal 3 month-old wild-type C57BL mouse fresh or fixed tissue samples from paraffin blocks were tested (n=10 for ovary, testis, spleen and skeletal muscle each).

### Cell Cultures

The murine spermatogenic GC-2(spd)ts (CRL-2196; ATCC, Manassas, VA), murine Leydig cell tumor (BLTK-1) (Kananen, Markkula et al. 1996) and Mouse Sertoli Cell line-1 (MSC-1) (Rebois 1982) cell lines (ATCC) were cultured in DMEM/F12 medium (GIBCO, Paisley, UK) supplemented with 10% (GC-2(spd)ts and BLTK-1) or 5% (MSC-1) fetal bovine serum (FBS; Biochrom, Berlin, Germany), 100 units/mL penicillin and 100 μg/mL streptomycin (P/S solution; Sigma-Aldrich, Saint Louis, MO) at 37° C. in a humidified atmosphere in the presence of 5% CO₂. The primary human Sertoli cell line HSerC (#4520; ScienCell Research Laboratories, Carlsbad, CA) was cultured in Sertoli Cell Medium (SerCM, #4521; ScienCell Research Laboratories), and the primary human Leydig cell line HLC (#4510; ScienCell Research Laboratories) in Leydig Cell Medium (LCM; #4511; ScienCell Research Laboratories) at 37° C. in a humidified atmosphere in the presence of 5% CO₂. Three independent cell platings in triplicates were performed for each RNA isolation and immunocytochemistry study.

### RNA Isolation

The TRIzol-based extraction method was used for total RNA isolation (Invitrogen, Carlsbad, CA). The quantity and quality of extracted RNA were determined by the absorbance measurement using NanoDrop (Thermo Scientific, Waltham, MA). Gel electrophoresis was performed to determine the integrity of isolated RNA.

### RT-PCR Analysis

Before the reverse transcription (RT) reaction, 1 μg of total RNA was incubated for 30 min with deoxyribonuclease (DNase I) (Invitrogen) at room temperature and inhibited by 25 mM EDTA solution for 10 min in 65° C. The RT reaction was performed with SensiFAST cDNA SynthesisKit (Bioline Reagents Ltd, London, UK) according to the manufacturer's protocol. First-strand cDNA was used as template in PCR (initial denaturation of 96° C. for 3 min, then 35 cycles of 94° C. for 1 min, 57° C. for 45 s, and 72° C. for 45 s, with a final extension period of 5 min at 72° C.). Primer sequences were as follows: mouse Zp3 gene F: GAGCTTTTCGGCATTTCAAG, R: AGCTTATCGGGGATCTGGTT and mouse Ppia gene F: CATCCTAAAGCATACAGGTCCTG, R: TCCATGGCTTCCACAATGTT; human
ZP3 gene F: ATGCAGGTAACTGACGATGC, R: CCATCAGACGCAGAGAGAAA,
human FSHR gene F: TGGGCTCAGGATGTCATCATCGGA,
R: TGGATGACTCGAAGCTTGGTGAGG, human LHR gene F: CTGAGTGGCTGGGACTATGA, R: CCAAATCAGGACCCTAAGGA; and human PPIA gene F: GCCAAGACTGAGTGGTTGGATG, R: GAGTTGTCCACAGTCAGCAATGG.
   Real-Time Quantitative PCR (qPCR)

For Real-time qPCR, SYBR Green PCR master mix (Applied Biosystems, Foster City, CA) and the thermo-cycler 7500 Real-Time PCR System (Applied Biosystems) were used. Reaction conditions were as follows: 2 min at 50° C., 10 min at 95° C., 15 s at 95° C., and 1 min at 60° C. up to 40 amplification cycles. PCR products were analyzed by melting curve analysis and agarose gel electrophoresis to ensure the amplification of a single product. Every reaction product (both for RT-PCR and qPCR) was separated and verified by sequencing analysis. Expression levels of the investigated genes were normalized to the housekeeping gene peptidylprolyl isomerase A (PPIA). The primer sequences were as follow: mouse Zp3 F: CCAACGACCAGACTGTGGAA, R: AGGACTATAGCTGCCAGGGT; mouse Ppia F: CATACAGGTCCTGGCATCTTGTC, R: AGACCACATGCTTGCCATCCAG; and human ZP3 F: TGGCAACAGCATGCAGGTA, R: CTGAGTGGCTGGGACTATGA, human FSHR F: GCCAAGAGAGCAAGGTGACA, R: CTCGAAGCTTGGTGAGGACA, human LHR F: CCGGTCTCACTCGACTATCACT, R: AAGCTTGAGATGGGATCACTTTG, and human PPIA F: GTTCTTCGACATTGCCGTCG, R: TGTCTGCAAACAGCTCAAAGG.

### RNAscope In Situ Hybridization

Formalin-fixed paraffin-embedded tissue samples were handled according to the manufacturer's protocol using RNAscope 2.0 HD Assay (catalog number 310033, Advanced Cell Diagnostics [ACD], Hayward, CA). In brief, slides were deparaffinized in xylene (2×5 min), 100% EtOH (2×1 min) and air-dried for 5 min at room temperature. Each section was treated with hydrogen peroxide for 10 min at RT, then washed twice in distilled water. Slides were boiled in antigen retrieval buffer for 15 min and submerged in distilled water immediately thereafter. Next, slides were washed in 100% EtOH and air-dried. For each section, barriers with hydrophobic pen were drawn and protease was applied for 30 min in 40° C. in HybEZ™ Oven (ACD). Slides were washed twice in distilled water. Probes for the targeted transcripts (mouse ZP3: ACD-447551, human ZP3: ACD-442631) were applied as well as probes for positive (cyclophilin B-PPIB, housekeeping gene, human ACD-313901; mouse ACD-313911) and negative controls (DapB-negative control probe targeting bacteria gene, ACD-310043). Then, slides were incubated at 40° C. for 2 h in the oven and washed 2×2 min in the wash buffer. Thereafter, hybridization amplifiers (AMPs) were applied for 30 min (AMP 1, 3, 5) or 15 min (AMP 2, 4, 6) at 40° C. (AMP 1-4) or at room temperature (AMP 5 and 6) with double washing in between every step. After the last washing, equal volumes of BROWN-A and BROWN-B reagents were combined and applied onto the sections for 10 min at RT. After double washing with distilled water, slides were counterstained in 50% Gill's hematoxylin (Vector Laboratories, Burlingame, CA, USA) for 2 min, then washed in 0.02% ammonia water for 10 s and twice in distilled water. Dehydrated slides (2×2 min in 70% EtOH, 2×2 min in 100% EtOH and 5 min in xylene) were mounted with Pertex (Histolab Products, Goteborg, Sweden).

### Immunohistochemical Staining

Monoclonal antibodies specific to human (Isotype IgG1 kappa) and mouse (Isotype IgG2a) ZP3 were produced with hybridoma techniques (East, Gulyas et al. 1985, Rankin, Tong et al. 1998). The hybridomas (ATCC® CRL-2462™ and ATCC® CRL-2569™) were cultured in CELLine bioreactor 1000 mL suspension flasks (Argos Technologies—Cole-Parmer, Vernon Hills, IL) in DMEM medium with 4 mM L-glutamine, 1.5 g/L sodium bicarbonate and 4.5 g/L glucose supplemented with 10 mM HEPES, 0.15 mg/ml oxaloacetate, 0.05 mg/ml pyruvate, 0.0082 mg/mL bovine insulin and 0.05 mM 2-mercaptoethanol. The 20% FBS was implemented in the cell compartment, and 1% FBS in the nutrient medium. Cells were harvested every −5 days, depending on their growth and then the harvests were purified.

Formalin- or Bouin-fixed paraffin-embedded samples were deparaffinized and hydrated. Slides were boiled for 15 min in antigen retrieval buffer (10 mM citric acid buffer with 0.05% Tween20, pH=6). Then, the sections were incubated in a humidified chamber for 1 h at RT with 3% BSA for reducing nonspecific background staining. Next, slides were incubated overnight in humidified chamber at 4° C. with the primary antibody anti-ZP3 (0.125 μg/mL) or anti-vimentin (ab28028; Abcam, Cambridge, UK; dilution 1:300). Endogenous peroxidase activity was blocked by incubating slides in 0.5% H₂O₂ in PBS at room temperature for 20 min. DAKO polymer (DAKO EnVision+System—HRP labelled polymer; Agilent, Santa Clara, CA) was applied onto each section and incubated in a humidified chamber for 30 min at room temperature. DAB+Chromogen (DAKO) was applied for 5 min. Slides were washed in dH₂O, counterstained in Mayer's hematoxylin (Sigma-Aldrich, Saint Louis, MO), dehydrated and mounted with Pertex (Histolab Products). As a control for the antibodies, tissues were incubated with 3% BSA and DAKO polymer to differentiate unspecific from specific staining.

### Immunocytochemical Staining

GC-2(spd)ts cells cultured on collagen-coated microscope slide coverslips were washed twice with PBS and fixed in 3.7% paraformaldehyde (PFA) for 20 min. For spermatozoa staining, sperm smear was made and allowed to air-dry at RT for a minimum of 1 hour. Next, slides with spermatozoa were fixed in 4% PFA in PBS for 1 hour. Slides were rinsed in PBS and treated with 0.5% Triton X-100 in PBS for 15 minutes. To block unspecific binding sites, cells were incubated in blocking solution (2% BSA in PBS with 0.05% Tween 20). Thereafter, cells were incubated for 1 h with primary mouse monoclonal anti-ZP3 antibody or for dual staining anti-ZP3 antibody with anti-α-tubulin antibody (ab52866, Abcam; dilution 1:300) diluted in blocking solution. After washing, cells were incubated with Alexa Fluor 488 goat anti-mouse IgG (A11029; Thermo Fisher, Waltham, MA; dilution 1:500) or Alexa Fluor 488 goat anti-rabbit IgG (A11034; Thermo Fisher; dilution 1:500) and Alexa Fluor 546 goat anti-mouse IgG (A11030; Thermo Fisher; dilution 1:500) for 45 min. DAPI dye was used as a counterstain to detect cell nuclei. As a control for the antibodies, the cells were incubated with either 2% BSA or Alexa Fluor-488 goat anti-mouse IgG as a primary antibody to differentiate unspecific from specific staining.

### Whole-Mount Mouse Seminiferous Tubule Staining

Whole-mount staining of adult mouse seminiferous tubules was performed as previously described (Makela, Cisneros-Montalvo et al. 2020). Briefly, three adult WT male mice were sacrificed and the tubules were fixed with 4% PFA for 2 hours at +4° C. After blocking (2% BSA+10% FBS in 0.3% Triton X-100 in PBS) the tubules were incubated overnight at +4° C. with primary antibodies: mouse monoclonal anti-human-ZP3 antibody (1 μg/ml), anti-GFRa1 antibody (AF560, R&D Systems, Minneapolis, MN; dilution 1:250) and anti-SALL4 antibody (1:2000, ab29112, Cambridge, UK). Following washes, the tubules were incubated with respective secondary antibodies (A11055, A10036 and A31573, Life Technologies, Carlsbad, CA, USA, dilution 1:500 for all) 1 hour at RT. Finally, the tubules were ordered into liner strips, mounted and imaged (Zeiss LSM880, Carl Zeiss, Jena, Germany).

### Results

Apart from the Ovary, ZP3 is Also Expressed in Normal Human and Wild-Type (WT) Mouse Testis in Spermatogenic Cells

Quantitative PCR analysis showed the Zp3 expression in WT mouse ovary and testis (FIG. 1A-1B, Table 1).

TABLE 1
Characteristics of ZP3 mRNA level in different
human and mouse tissues/cells.

| | | | | BLTK-1/ | | |
|---|---|---|---|---|---|---|
| Tissue | OV | T | GC-2spd(ts) | MSC-1 | HSerC/HLC | SP/MU |
| ZP3 | +++ | ++ | ++ | − | − | − |

mRNA:
−, not detected;
++, 25-30 cycles;
+++, 20-25 cycles
BLTK-1, murine Leydig cell tumor;
GC-2spd(ts), mouse spermatogenic cell line;
HLC, primary human Leydig cell line;
HSerC, primary human Sertoli cell line;
MSC-1, Mouse Sertoli Cell line-1;
MU, muscle;
OV, ovary;
SP, spleen;
T, testis;

Zp3 expression was not detectable in WT mouse spleen and muscle used as negative controls (FIG. 1A-1B). Immunohistochemical staining and RNAscope in situ hybridization localized abundant Zp3 protein and mRNA transcripts in oocytes from primary, secondary and antral follicles of the mouse ovary (FIGS. 2A and 2C, respectively). Similarly, abundant Zp3 protein and mRNA were localized in spermatogonia, spermatocytes, round and elongated spermatids in the mouse testis (FIGS. 2B and 2D, respectively). The mouse primordial follicle oocytes and testicular Sertoli and Leydig cells, as well as the spermatozoa did not express Zp3 (FIGS. 2A-2D). In humans, abundant ZP3 protein and mRNA transcripts were also localized in the oocytes from ovarian follicles (FIGS. 3A and 3C, respectively) and in testicular spermatogonia, spermatocytes, round and elongated spermatids (FIGS. 3B and 3D, respectively). ZP3 expression was not detected in the human testicular Sertoli and Leydig cells, either at mRNA or protein levels (FIGS. 3B and 3D). Cyclophilin B was used as a positive control, which was expressed abundantly in mouse and human ovary and testis (FIG. 4, left column). DapB was used as a negative control, and it was not detected in any of the gonadal samples (FIG. 4, right column). The omission of the primary antibody was used as a control for immunohistochemical analysis, and no staining in mouse and human testis was detected (FIG. 5 A-B). To identify Sertoli cells testicular tissue was immunostained with vimentin, a Sertoli cell marker. Positive immunoreactivity was observed for vimentin in the cytoplasm of Sertoli cells with a thin layer of the cytoplasm passing through the whole epithelium (FIG. 6 A-B). ZP3 mRNA transcripts were not found in human primordial follicle oocytes (FIG. 7A), whereas abundant expression was localized in oocytes from primary (FIG. 7B), secondary (FIG. 7C) and antral (FIG. 7D) follicles. Zp3 expression was also found in the mouse GC-2spd(ts) immortalized spermatogenic cell line and WT mouse ovary as positive control (FIG. 8A). No Zp3 expression was detected in Leydig tumor cell line BLTK-1 and Sertoli MSC-1 cells (FIG. 8A). WT mouse skeletal muscle was used as Zp3 negative control (FIG. 8A). Immunocytochemical studies localized Zp3 expression in the cytoplasms of mouse GC-2spd(ts) cells (FIG. 8B). No staining was detected in the control analysis without a primary antibody (FIG. 8B). ZP3 mRNA expression was not either detectable in human primary Sertoli (HSerC) and Leydig (HLC) cells (FIG. 9A-9B). FSHR and LHR expression were analyzed as a control for HSerC and HLC cells, respectively. HSerC expressed FSHR and no LHR, whereas HLC expressed LHR and no FSHR (FIG. 9A-9B). ZP3, LHR, FSHR expression were found in human ovary used as a positive control, whereas was not detectable in human muscle used as a negative control (FIG. 9A-9B). GFRa1-positive (GDNF family receptor alpha-1) spermatogonia are considered stem and progenitor cells (SSPCs) (differentiating spermatogonia, cells that precede A1 spermatogonia that are differentiated and committed to sperm development) of the male germline and thus responsible for the life-long sperm production. In order to check Zp3 expression in SSPCs, a whole-mount seminiferous tubule staining for adult mouse testis was performed. Immunoreactivity was not observed for mouse monoclonal anti-ZP3 antibody on the basement membrane of adult mouse seminiferous epithelium suggesting that GFRa1/SALL4-positive (Spalt-like 4, a pan-spermatogonial marker) SSPCs and differentiating progenitor spermatogonia (GFRa1-negative/SALL4-positive) do not express ZP3 (FIG. 10). Neither was ZP3 mRNA expression detectable in human spermatozoa (FIG. 11). ZP3 expression was found in the human ovary used as a positive control, whereas it was not detectable in human muscle used as a negative control (FIG. 11A). No ZP3 staining was detected using immunocytochemical analysis in human spermatozoa (FIG. 11B).

Various Other Normal Human and Mouse Tissues were ZP3 Negative

ZP3 expression was not detected by immunohistochemical staining in several other normal human tissues, such as the small intestine (FIG. 12a), large intestine (FIG. 12b), liver (FIG. 12c), pancreas (FIG. 12d), stomach (FIG. 12e), lymph node (FIG. 12f), brain (FIG. 12g), lung (FIG. 12h), epididymis (FIG. 12i), prostate (FIG. 12j), kidney (FIG. 12k), breast (FIG. 12l), smooth muscle myometrium (FIG. 12m) and skeletal muscle (FIG. 12n).

### DISCUSSION

ZP3 has been shown to be expressed in several cancers (ovarian, prostate, colon and lung), giving rise to the concept of its use as a target for cancer immunotherapy. It has also been shown that ZP3 immunization is an effective treatment of ovarian cancer, using a transgenic mouse model of ovarian granulosa cell tumors. The immunization strategy used has the potential of being effective in the treatment of other cancer types expressing ZP3. The present study was designed to characterize ZP3 expression in healthy tissues, with the potential of off-target effects in cancer immunization.

The present results show the novel expression of ZP3 in spermatogonia, spermatocytes and spermatids in the human and mouse testis. However, no expression was found in mature spermatozoa, spermatogonial stem and progenitor cells and in Sertoli or Leydig cells. In the ovary, ZP3 is required for successful oocyte fertilization, but there is no data on any ZP3 function outside the ovary. Protein ZP3 expression was not found in any other healthy normal tissues, except for the ovary and the testis.

In the present work, several techniques were used to check/confirm the ZP3 expression. The ZP3 protein localization was analyzed with a specific monoclonal antibody and confirmed its presence by RNA transcripts in the testis using a sensitive RNAscope in situ hybridization method.

In female animals, ZP3 immunization induces autoimmune oophoritis and atrophy of the ovaries (Lou, Y. H., K. K. Park, S. Agersborg, P. Alard and K. S. Tung (2000). “Retargeting T cell-mediated inflammation: a new perspective on autoantibody action.” J Immunol 164(10): 5251-5257). When primordial ovarian follicles are selected for growth and become primary follicles, they start to express ZP antigens in the glycoprotein layer around the oocyte and become susceptible to ZP3-specific antibodies and autoreactive immune cells. It has been demonstrated that antibody binding to ZP3 protein in growing or mature follicles is followed by induction of T cell-mediated inflammation resulting in degeneration of developing follicles. Recently, it has been shown that in actively immunized mice after careful selection of B-cell epitopes, ZP3 vaccines may induce reversible infertility without adverse effects on the ovaries (Paterson, M., Z. A. Jennings, M. R. Wilson and R. J. Aitken (2002). “The contraceptive potential of ZP3 and ZP3 peptides in a primate model.” J Reprod Immunol 53(1-2): 99-107). With a chimeric peptide vaccine, the defined B-cell epitope had a single critical amino acid substitution to prevent cross-reaction with a native ZP3 T-cell epitope, but not B-cell response to ZP3 (Lou, Y., J. Ang, H. Thai, F. McElveen and K. S. Tung (1995). “A zona pellucida 3 peptide vaccine induces antibodies and reversible infertility without ovarian pathology.” J Immunol 155(5): 2715-2720). This ZP3 vaccine was able to successfully prevent pregnancy without causing ovarian pathology.

ZP3 antigen expression in the testis introduces the surprising possibility to use ZP3 immunization for male contraception. The absence of ZP3 expression in any other normal male tissues, as well as in somatic testicular cells, reduces the risk of concomitant damage to other organs. However, the ZP3 epitopes should be carefully selected to avoid the destruction of testicular tissue and allow the ability to cross the blood-testis barrier. Another important feature of such immunization would be its reversibility. Also, since ZP3 is not expressed by the testosterone synthesizing Leydig cells in the testis, unfavorable endocrine side effects may not occur, provided no general auto-immune response occurs due to adequate selection of the epitopes. Furthermore, since ZP3 expression was not detected in the SSPCs, as well as in Sertoli cells, it is unlikely that anti-ZP3 male contraceptive therapy would destroy spermatogenic stem cells to induce irreversible infertility; hence this male contraceptive strategy could be reversible upon discontinuation.

In conclusion, the novel extraovarian ZP3 expression in human and mouse spermatogenic cells, and the absence of ZP3 expression in a series of normal human and mouse tissues is reported here. These findings on absent ZP3 expression in normal nongonadal tissues provide crucial background information for any ZP3 cancer immunotherapy. Any future ZP3 cancer immunotherapy clinical trials in males (such as prostate, lung or colorectal cancer) should have a caution due to the ZP3 expression in testis. Besides cancer immunotherapy, another therapeutic strategy was proposed by the finding on ZP3 expression in the testis. The exclusive localization of testicular ZP3 expression in spermatogonia, spermatocytes and spermatids make ZP3 immunization a promising new target for male immunocontraception. Further studies should also address the functional role of ZP3 in the testis, and in particular whether ZP3 immunization has a long-term or reversible effect on male fertility.

### DESCRIPTION OF THE FIGURES

FIG. 1: Zp3 expression profile in different wild-type (WT) mouse tissues. The qPCR analysis of Zp3 expression in normal mouse testis, ovary, spleen and muscle (A). Expression of Zp3 was normalized to that of Ppia expression in the same sample. The RT-PCR analysis in 2% agarose gel of housekeeping gene Ppia and Zp3 in normal mouse testis, ovary, spleen and muscle (B). Amplicon sizes are presented on the left. M, marker; MU, muscle; OV, ovary; SP, spleen; TE, testis; H₂O, nuclease-free water.

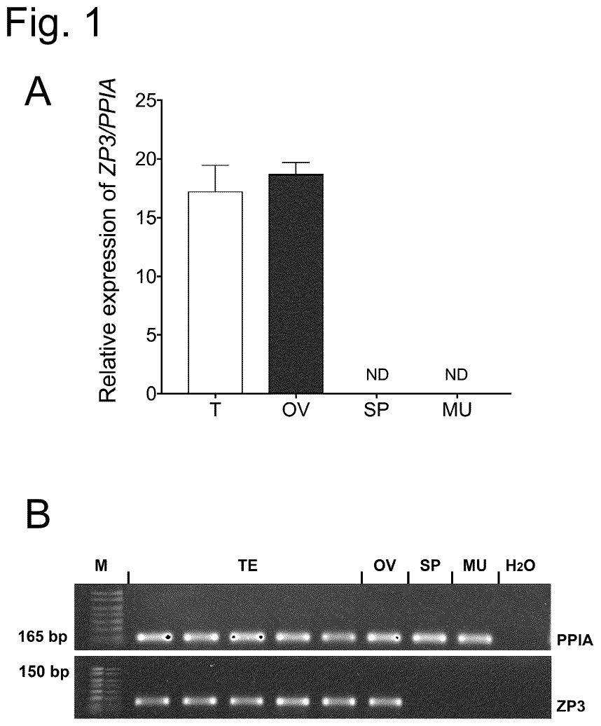

FIG. 2: Immunohistochemical localization and RNAscope in situ hybridization of Zp3 in wild-type (WT) mouse ovary and testis. Localization of Zp3 protein in mouse ovary (A) and testis (B) and Zp3 mRNA transcripts in mouse ovary (C) and testis (D). The upper box on the right shows higher magnification of the lower box, showing ZP3 protein/transcripts localization in mouse testis (B, D). Black arrow-heads show the positive ZP3 protein staining or single transcripts localization, white arrow-head indicates negative Sertoli cells and blue arrow-head shows negative Leydig cells in mouse testis. Scale bar, 100 μm (A, C) and 50 μm (B, D).

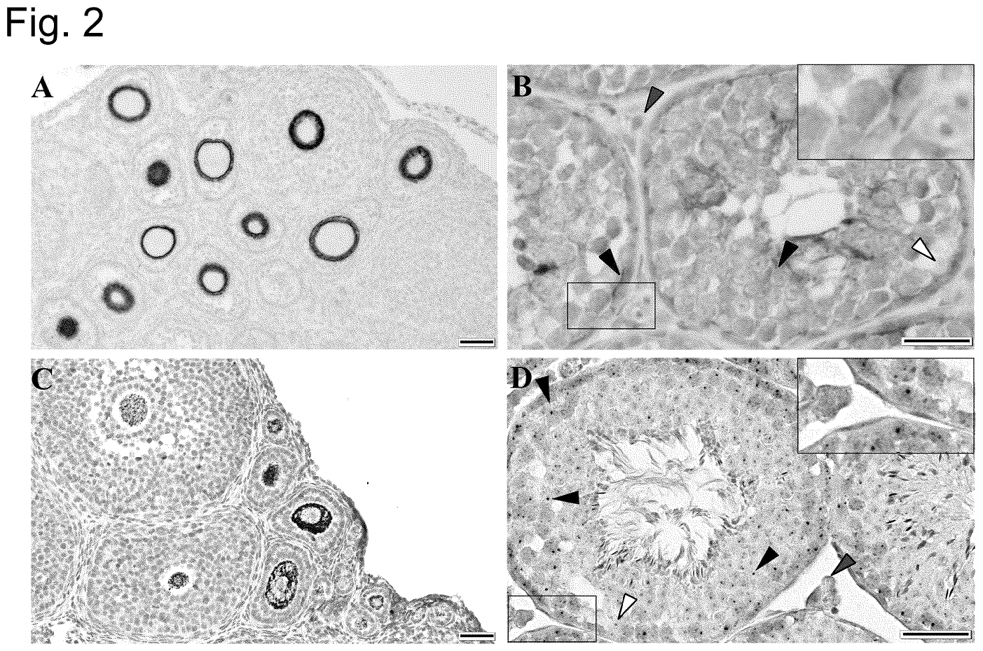

FIG. 3: Immunohistochemical localization and RNAscope in situ hybridization of ZP3 mRNA transcripts in normal human ovary and testis. Localization of ZP3 protein in the human ovary (A) and testis (B) and ZP3 mRNA transcripts in the human ovary (C) and testis (D). The upper box on the right shows higher magnification of the lower box, showing ZP3 protein/transcripts localization in human testis (B, D). Black arrow-heads show the positive ZP3 protein staining or single transcripts localization, white arrow-head indicates negative Sertoli cells and blue arrow-head shows negative Leydig cells in human testis. Scale bar, 100 μm (A, B) and 50 μm (C, D).

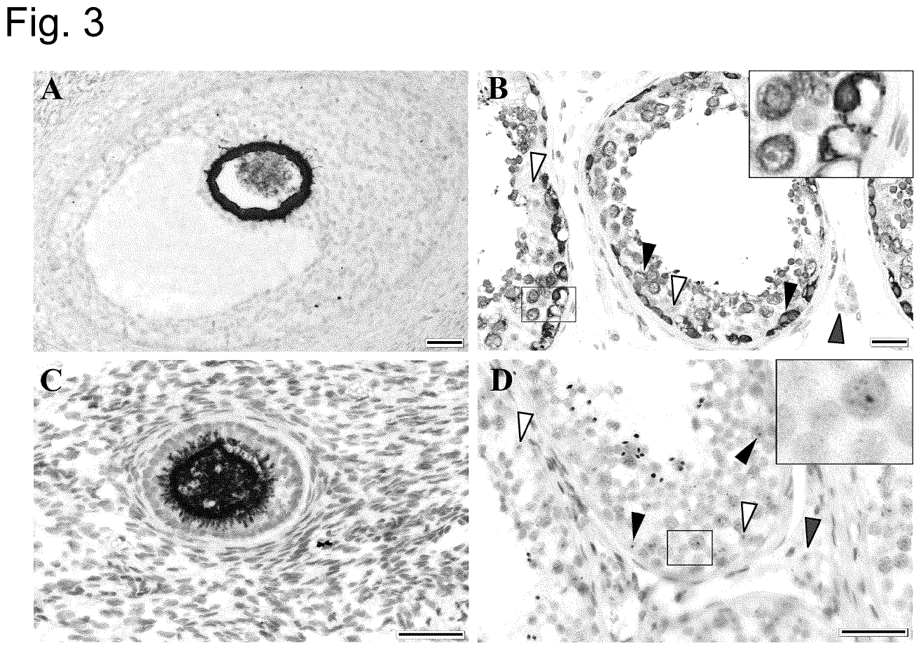

FIG. 4: Analysis of tissue sections quality and the specificity of RNAscope® in situ hybridization. Paraffin sections of wild-type mouse ovary (A-B) and human normal ovary (C-D) and mouse normal testis (E-F) and human normal testis (G-H) were hybridized with a positive control probe complementary to Cyclophilin B (left column) and negative control probe that targets bacteria DapB gene (right column). Sections were counterstained with hematoxylin. Scale bar, 50 μm.

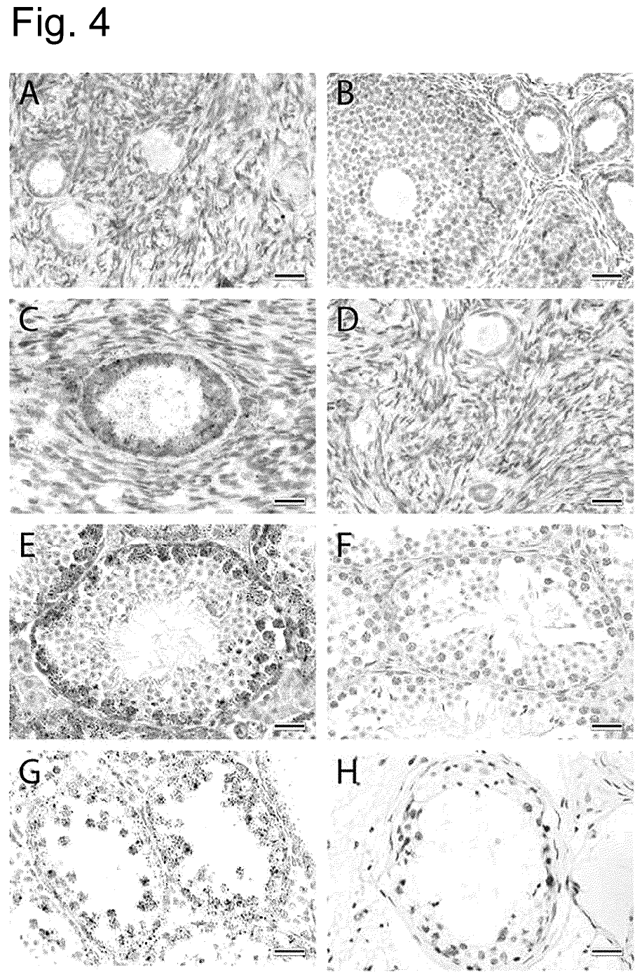

FIG. 5: Analysis of the primary antibody specificity in immunohistochemical studies. Paraffin sections of wild-type mouse testis (A) and human testis (B) were incubated with 3% BSA and DAKO EnVision+System-HRP labelled polymer. Sections were counterstained with hematoxylin. Scale bar, 50 μm.

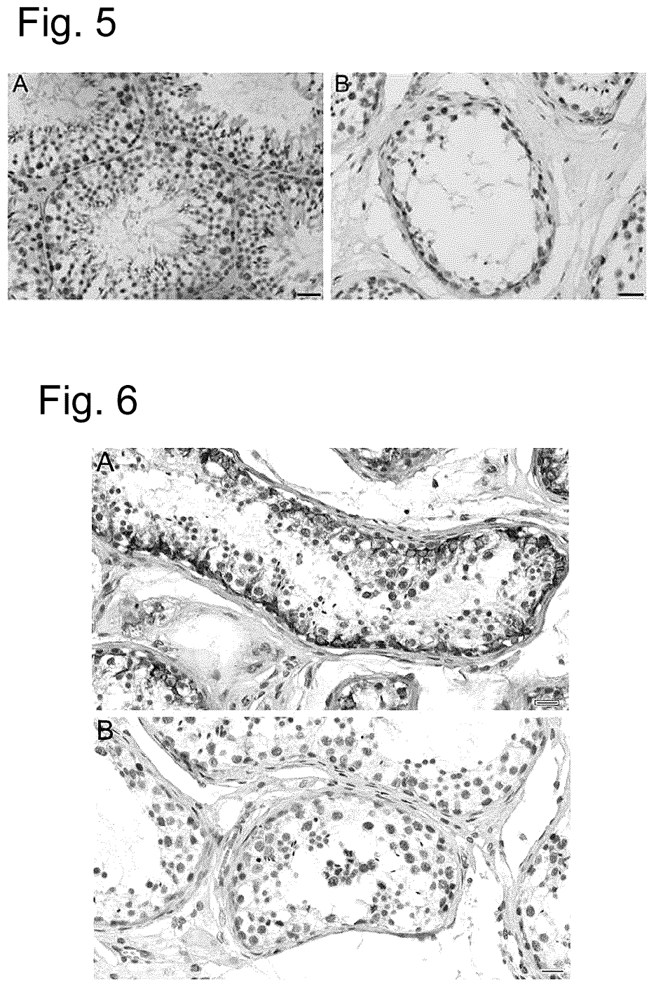

FIG. 6: Immunohistochemical localization of the vimentin, Sertoli cells marker. Localization of vimentin in mouse (A) and human (B) testis. Analysis of the primary antibody specificity in mouse (C) and human (D) testis. Scale bar, 50 μm.

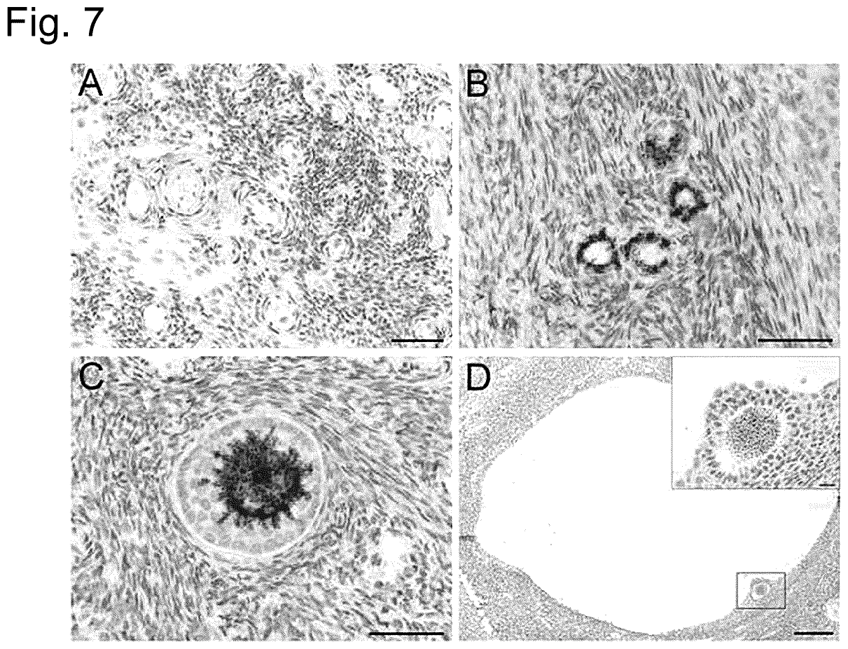

FIG. 7: RNAscope in situ hybridization of ZP3 mRNA transcripts in the normal human ovary. Localization of ZP3 mRNA transcripts in oocytes from primordial (A), primary (B), secondary (C) and antral (D) follicles. The upper box on the right shows higher magnification of the lower box, revealing ZP3 localization in oocyte from the antral follicle (D). Scale bar, 100 μm (A), 50 μm (B, C) and 200 μm (D). Box magnification, 40×; scale bar, 50 μm.

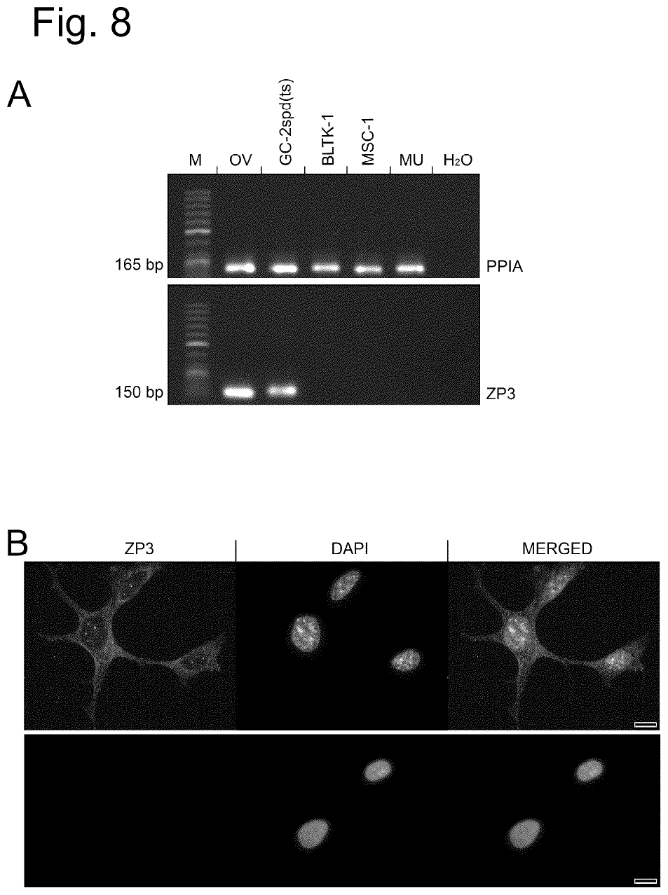

FIG. 8: Zp3 expression in mouse GC-2spd(ts) cell line. The RT-PCR analysis in 2% agarose gel of housekeeping gene Ppia and Zp3 in GC-2spd(ts) cell line, BLTK-1 cell line, MSC-1 cell line, ovary and muscle (A). Amplicon sizes are presented on the left. Immunocytochemical localization of Zp3 in mouse spermatogenic GC-2spd(ts) cell line (B). The lower panel shows no primary antibody control staining. BLTK-1, murine Leydig cell tumor; GC-2spd(ts), mouse spermatogenic cell line; M, marker; MSC-1, Mouse Sertoli Cell line-1; MU, muscle; OV, ovary; H₂O, nuclease-free water. Scale bar, 10 μm.

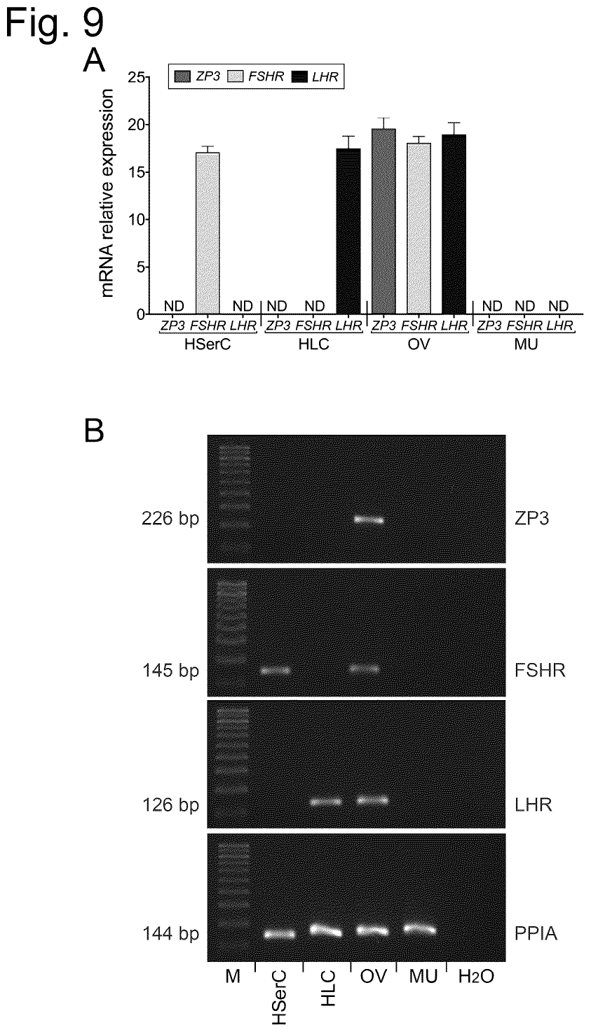

FIG. 9: ZP3, FSHR and LHR expression profiles in human primary Sertoli and Leydig cells. The qPCR analysis of ZP3, FSHR and LHR expression in human primary HSerC and HLC cells and normal human ovary and muscle (A). The expression of ZP3 was normalized to that of PPIA expression in the same sample. The RT-PCR analysis in 2% agarose gel of ZP3, FSHR, LHR and housekeeping gene PPIA in human primary HSerC and HLC cells and normal human ovary and muscle (B). HLC, primary human Leydig cell line; HSerC, primary human Sertoli cell line; M, marker; MU, muscle; OV, ovary; H₂O, nuclease-free water.

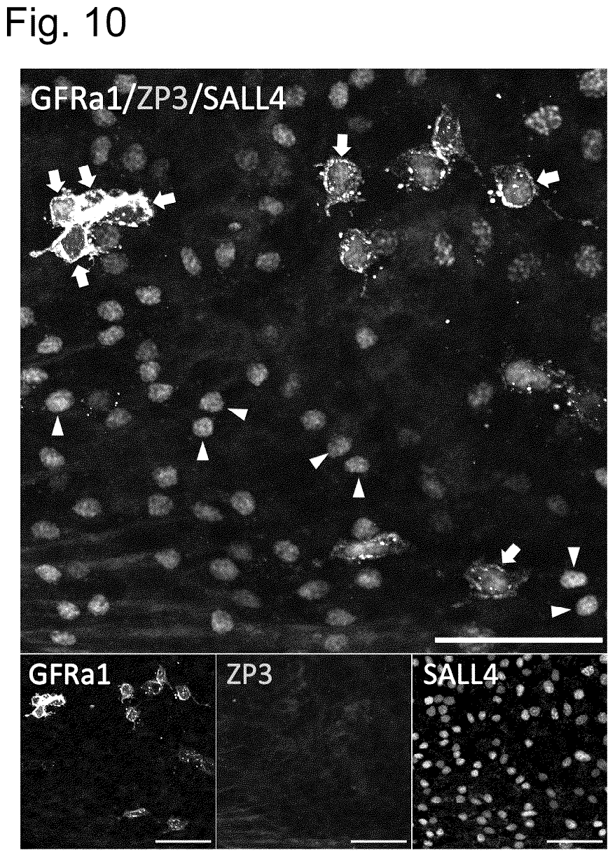

FIG. 10: Immunohistochemical localization of ZP3 in mouse spermatogonial stem and progenitor cells, SSPCs. A confocal microscopy image of adult mouse whole-mount seminiferous tubule staining using antibodies against SSPC marker GFRa1 (red), ZP3 (green) and a pan-spermatogonial marker SALL4 (blue). White arrows indicate GFRa1/SALL4-positive SSPCs, white arrowheads point at GFRa1-negative/SALL4-positive differentiating progenitor spermatogonia. Scale bars 50 μm.

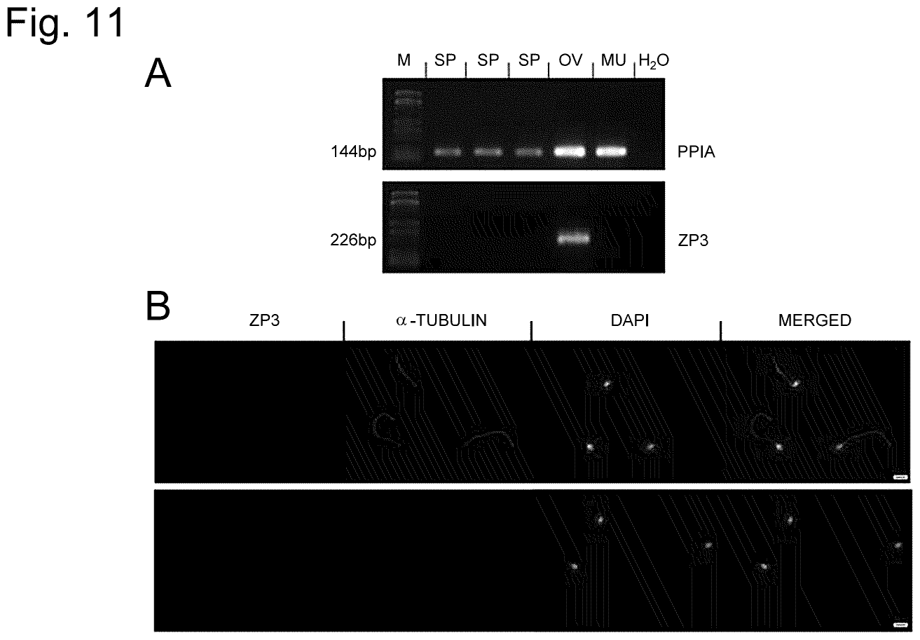

FIG. 11: ZP3 expression in human spermatozoa. The RT-PCR analysis in 2% agarose gel of housekeeping gene PPIA and ZP3 in human spermatozoa, ovary and muscle (A). Amplicon sizes are presented at the left. Immunocytochemical localization of ZP3 and α-tubulin in human spermatozoa (B). The lower panel shows no primary antibody control staining. M, marker; MU, muscle; OV, ovary; SP, spermatozoa; H₂O, nuclease-free water. Scale bar, 10 μm

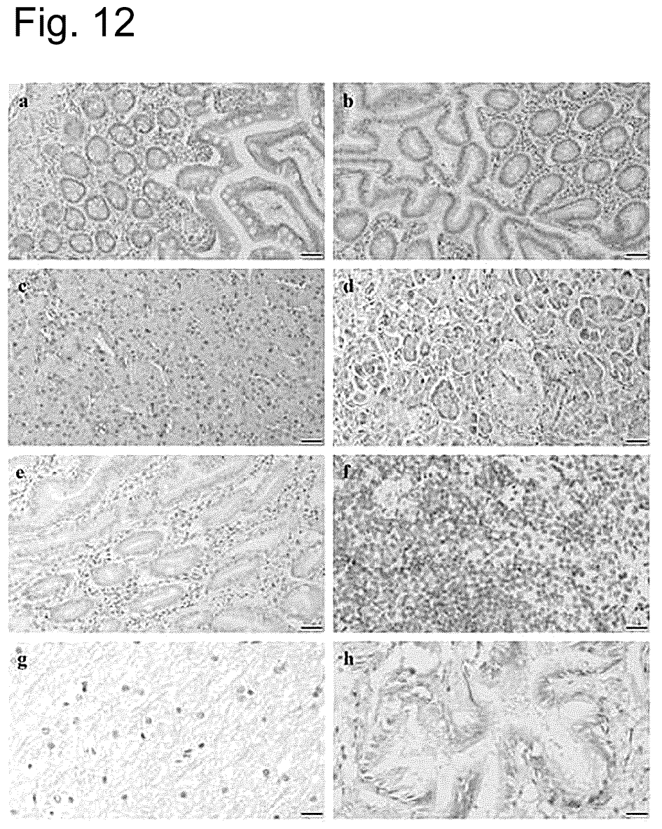

FIG. 12: Immunohistochemical localization of ZP3 in several normal human tissues. Localization of ZP3 protein in human small intestine (a), large intestine (b), liver (c), pancreas (d), stomach (e), lymph node (f), brain (g), lung (h), epididymis (i), prostate (j), kidney (k), breast (l), smooth muscle myometrium (in) and skeletal muscle (n). Scale bar, 50 μm.

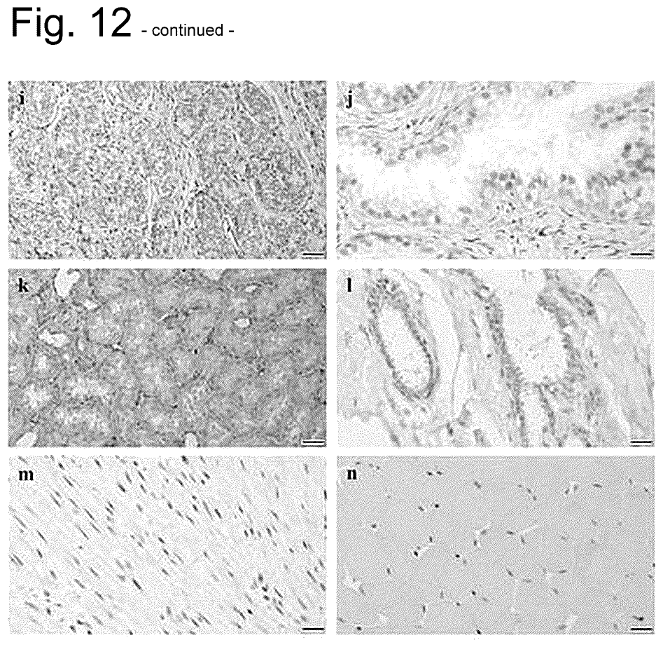

FIG. 13: Selected hZP3 peptide sequences. Boxes and ovals enclose predicted MHC class I and II binders as indicated in Tables 2 and 5, and 3, respectively. Asterisks indicate potential N-inked glycosylation sites, and the brackets above the sequences mark regions with cluster of O-linked glycans.

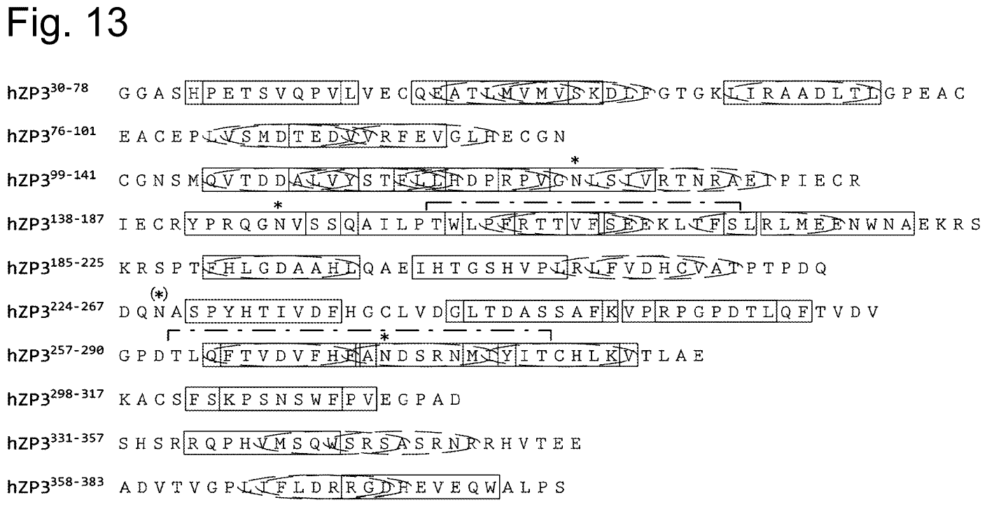

### Tables

TABLE I
predicted MHC class I ligands contained in the
hZP3 protein:

| | | | SEQ |
|---|---|---|---|
| | Ligand | | ID |
| aa* | sequence | HLA allele (% Rank) | NO: |
| 34-42 | HPETSVQPV | HLA-B*07:02 (0.80) | 6 |
| 35-43 | PETSVQPVL | HLA-B*40:01 (0.80) | 7 |
| 47-55 | ***QEATLMVMV | HLA-B*40:01 (0.80) | 8 |
| 49-57 | ATLMVMVSK | HLA-A*03:01 (0.30) | 9 |
| 65-73 | LIRAADLTL | HLA-B*07:02 (0.80) | 10 |
| 86-94 | TEDVVRFEV | HLA-B*40:01 (0.40) | 11 |
| 104-112 | QVTDDALVY | HLA-A*01:01 (0.30); | 12 |
| | | HLA-A*26:01 (0.40) | |
| 109-117 | ALVYSTFLL | HLA-A*02:01 (0.80) | 13 |
| 115-123 | FLLHDPRPV | HLA-A*02:01 (0.80) | 14 |
| 121-129 | RPVGNLSIV^{¶} | HLA-B*07:02 (0.30) | 15 |
| 142-150 | YPRQGNVSS^{¶} | HLA-B*07:02 (0.80) | 16 |
| 149-157 | SSQAILPTW | HLA-B*58:01 (0.10) | 17 |
| 152-160 | AILPTWLPF | HLA-B*15:01 (0.80) | 18 |
| 157-165 | WLPFRTTVF | HLA-A*24:02 (0.80), | 19 |
| | | HLA-3*08:01, | |
| | | HLA-3*15:01 (0.80) | |
| 164-172 | VFSEEKLTF | HLA-A*24:02 (0.30) | 20 |
| 166-174 | SEEKLTFSL | HLA-3*40:01 (0.15) | 21 |
| 175-183 | RLMEENWNA | HLA-A*02:01 (0.20) | 22 |
| 190-198 | FHLGDAAHL | HLA-3*39:01 (0.05) | 23 |
| 202-210 | ***IHTGSHVPL | HLA-3*39:01 (0.10) | 24 |
| 228-234 | SPYHTIVDF | HLA-3*07:02 (0.80) | 25 |
| 243-251 | GLTDASSAF | HLA-3*15:01 (0.20) | 26 |
| 244-252 | LTDASSAFK | HLA-A*03:01 (0.80) | 27 |
| 253-261 | VPRPGPDTL | HLA-3*07:02 (0.15) | 28 |
| 255-263 | RPGPDTLQF | HLA-3*07:02 (0.30) | 29 |
| 262-270 | QFTVDVFHF | HLA-A*24:02 (0.80) | 30 |
| 263-271 | FTVDVFHFA | HLA-A*02:01 (0.80), | 31 |
| | | HLA-A*26:01 (0.80) | |
| 271-279 | ANDSRNMIY^{¶} | HLA-A*01:01 (0.30) | 32 |
| 277-285 | MIYITCHLK | HLA-A*03:01 (0.10) | 33 |
| 278-286 | IYITCHLKV | HLA-A*24:02 (0.20) | 34 |
| 302-310 | FSKPSNSWF | HLA-3*15:01 (0.80) | 35 |
| 304-312 | KPSNSWFPV | HLA-3*07:02 (0.30) | 36 |
| 334-342 | RQPHVMSQW | HLA-A*24:02 (0.80) | 37 |
| 371-379 | RGDHEVEQW | HLA-3*58:01 (0.80) | 38 |

*Position is that of aa in complete hZP3 protein.
^{¶}Ligand contains N-linked glycosylation site (position 125, 147 and 272, respectively).

TABLE II
predicted MHC class II ligands contained in the hZP3 protein:

| aa* | Ligand sequence | HLA allele (percentile rank^{§}) | SEQ ID NO: |
|---|---|---|---|
| 48-56 | EATLMVMVS | DQA1*01:02/DQB*06:02 (0.14) | 39 |
| 51-59 | LMVMVSKDL | DRB5*01:01 (0.88); | 40 |
| | | DRB1*15:01 (2.37) | |
| 52-60 | MVMVSKDLF | DRB1*03:01 (1.01) | 41 |
| 65-73 | ****LIRAADLTL | DRB1*08:02 (2.49) | 42 |
| 81-89 | LVMSDTEDV | DRB1*01:02 (1.31) | 43 |
| 82-90 | VSMDTEDVV | DRB1*03:01 (0.54) | 44 |
| 89-97 | VVRFEVGLH | DRB1*11:02 (0.52) | 45 |
| 104-112 | QVTDDALVY | DRB1*03:01 (1.91) | 46 |
| 109-117 | ALVYSTFLL | DPA1*01:03/DPB1*02:01 (0.01); | 47 |
| | | DPA1*02:01/DPB1*01:01 (0.12) | |
| 110-118 | LVYSTFLLH | DPA1*01/DPB1*04:01 (0.01); | 48 |
| | | DPA1*0301/DPB1*04:02 (0.04) | |
| 115-123 | FLLDPHRPV | DRB1*11:02 (0.39) | 49 |
| 116-124 | LLHDPRPVG | DRB1*03:01 (0.17) | 50 |
| 126-134 | *LSIVRTNRA^{‡} | DRB1*08:02 (1.55) | 51 |
| 128-136 | IVRTNRAEI | DRB1*03:01 (1.77) | 52 |
| 159-167 | PFRTTVFSE | DPA1*01:03/DPB1*02:01 (0.58); | 53 |
| | | HLA-DPA1*02:01/DPB1*01:01 (0.69) | |
| 160-168 | FRTTVFSEE | DPA1*01/DPB1*04:01 (0.35) | 54 |
| 171-179 | **TFSLRLMEE | DQA1*04:01/DQB1*04:02 (1.05) | 55 |
| 190-198 | FHLGDAAHL | DRB1*09:01 (0.49) | 56 |
| 210-218 | LRLFVDHCV | DQA1*01:01 (0.48) | 57 |
| 212-220 | LFVDHCVAT | DRB1*03:01 (2.05) | 58 |
| 262-270 | QFTVDVFHF | DRB3*01:01 (1.5) | 59 |
| 270-278 | FANDSNRMI^{¶} | DRB3*01:01 (0.35); DRB3*02:02 | 60 |
| | | (1.37); DRB1*03:01 (1.77) | |
| 278-286 | IYITCHLKV | DRB1*11:01 (1.16); | 61 |
| | | DPA1*03:01/DPB1*04:02 (1.53) | |
| 339-347 | VMSQWSRSA | DRB1*11:02 (0.79) | 62 |
| 343-351 | WSRSASRNR | DRB5*01:01 (1.4) | 63 |
| 365-373 | LIFLDRRGD | DRB1*11:02 (1.01) | 64 |
| 366-374 | IFLDRRGDH | DRB1*03:01 (0.08) | 65 |

*Position is that of aa in complete hZP3 protein.
^{§}The percentile rank is given in parentheses after the corresponding HLA allele; only ligands with percentile ranks >2.5 are considered.
^{‡}N-linked glycosylation site in preceding position 125.
^{¶}Ligand contains N-linked glycosylation site (position 272).

TABLE III
HLA-A2 restricted T cell epitopes from rhZP3 protein:

| No. | Position | Peptide | Affinity (nM) | % Rank | SEQ ID NO. |
|---|---|---|---|---|---|
| 1 | 115 | FLLHDPRPV | 5.1 | 0.04 | 76 |
| 2 | 175 | RLMEENWNA | 5.2 | 0.04 | 77 |
| 3 | 109 | ALVYSTFLL | 21.3 | 0.3 | 78 |
| 4 | 263 | FTVDVFHFA | 19.9 | 0.3 | 79 |
| 5 | 174 | LRLMEENWNA | 28.6 | 0.4 | 80 |
| 6 | 367 | FLDRRGDHEV | 53.2 | 0.7 | 81 |
| 7 | 239 | CLVDGLTDA | 79.1 | 0.9 | 82 |
| 8 | 276 | NMIYITCHL | 76.6 | 0.9 | 83 |
| 9 | 114 | TFLLHDPRPV | 101.9 | 1.1 | 84 |
| 10 | 64 | KLIRAADLTL | 129.9 | 1.2 | 85 |

TABLE IV
human zona pellucida proteins:

| # | Sequence |
|---|---|
| SEQ ID NO: 1 | MAGGSATTWGYPVALLLLVATLGLGRWLQPDPGLPGLRHSYDCGIKGMQLLVFPRPGQTLRFKVVDEFG |
| | NRFDVNNCSICYHWVTSRPQEPAVFSADYRGCHVLEKDGRFHLRVFMEAVLPNGRVDVAQDATLICPKP |
| | DPSRTLDSQLAPPAMFSVSTPQTLSFLPTSGHTSQGSGHAFPSPLDPGHSSVHPTPALPSPGPGPTLAT |
| | LAQPHWGTLEHWDVNKRDYIGTHLSQEQCQVASGHLPCIVRRTSKEACQQAGCCYDNTREVPCYYGNTA |
| | TVQCFRDGYFVLVVSQEMALTHRITLANIHLAYAPTSCSPTQHTEAFVVFYFPLTHCGTTMQVAGDQLI |
| | YENWLVSGIHIQKGPQGSITRDSTFQLHVRCVFNASDFLPIQASIFPPPSPAPMTQPGPLRLELRIAKD |
| | ETFSSYYGEDDYPIVRLLREPVHVEVRLLQRTDPNLVLLLHQCWGAPSANPFQQPQWPILSDGCPFKGD |
| | SYRTQMVALDGATPFQSHYQRFTVATFALLDSGSQRALRGLVYLFCSTSACHTSGLETCSTACSTGTTR |
| | QRRSSGHRNDTARPQDIVSSPGPVGFEDSYGQEPTLGPTDSNGNSSLRPLLWAVLLLPAVALVLGFGVF |
| | VGLSQTWAQKLWESNRQ |
| SEQ ID NO: 2 | MACRQRGGSWSPSGWFNAGWSTYRSISLFFALVTSGNSIDVSQLVNPAFPGTVTCDEREITVEFPSSPG |
| | TKKWHASVVDPLGLDMPNCTYILDPEKLTLRATYDNCTRRVHGGHQMTIRVMNNSAALRHGAVMYQFFC |
| | PAMQVEETQGLSASTICQKDFMSFSLPRVFSGLADDSKGTKVQMGWSIEVGDGARAKTLTLPEAMKEGF |
| | SLLIDNHRMTFHVPFNATGVTHYVQGNSHLYMVSLKLTFISPGQKVIFSSQAICAPDPVTCNATHMTLT |
| | IPEFPGKLKSVSFENQNIDVSQLHDNGIDLEATNGMKLHFSKTLLKTKLSEKCLLHQFYLASLKLTFLL |
| | RPETVSMVIYPECLCESPVSIVTGELCTQDGFMDVEVYSYQTQPALDLGTLRVGNSSCQPVFEAQSQGL |
| | VRFHIPLNGCGTRYKFEDDKVVYENEIHALWTDFPPSKISRDSEFRMTVKCSYSRNDMLLNINVESLTP |
| | PVASVKLGPFTLILQSYPDNSYQQPYGENEYPLVRFLRQPIYMEVRVLNRDDPNIKLVLDDCWATSTMD |
| | PDSFPQWNVVVDGCAYDLDNYQTTFHPVGSSVTHPDHYQRFDMKAFAFVSEAHVLSSLVYFHCSALICN |
| | RLSPDSPLCSVTCPVSSRHRRATGATEAEKMTVSLPGPILLLSDDSSFRGVGSSDLKASGSSGEKSRSE |
| | TGEEVGSRGAMDTKGHKTAGDVGSKAVAAVAAFAGVVATLGFIYYLYEKRTVSNH |
| SEQ ID NO: 3 | MELSYRLFICLLLWGSTELCYPQPLWLLQGGASHPETSVQPVLVECQEATLMVMVSKDLFGTGKLIRAA |
| | DLTLGPEACEPLVSMDTEDVVRFEVGLHECGNSMQVTDDALVYSTFLLHDPRPVGNLSIVRTNRAEIPI |
| | ECRYPRQGNVSSQAILPTWLPFRTTVFSEEKLTFSLRLMEENWNAEKRSPTFHLGDAAHLQAEIHTGSH |
| | VPLRLFVDHCVATPTPDQNASPYHTIVDFHGCLVDGLTDASSAFKVPRPGPDTLQFTVDVFHFANDSRN |
| | MIYITCHLKVTLAEQDPDELNKACSFSKPSNSWFPVEGSADICQCCNKGDCGTPSHSRRQPHVMSQWSR |
| | SASRNRRHVTEEADVTVGPLIFLDRRGDHEVEQWALPSDTSVVLLGVGLAVVVSLTLTAVILVLTRRCR |
| | TASHPVSASE |
| SEQ ID NO: 4 | MWLLRCVLLCVSLSLAVSGQHKPEAPDYSSVLHCGPWSFQFAVNLNQEATSPPVLIAWDNQGLLHELQN |
| | DSDCGTWIRKGPGSSVVLEATYSSCYVTEWDSHYIMPVGVEGAGAAEHKVVTERKLLKCPMDLLARDAP |
| | DTDWCDSIPARDRLPCAPSPISRGDCEGLGCCYSSEEVNSCYYGNTVTLHCTREGHFSIAVSRNVTSPP |
| | LLLDSVRLALRNDSACNPVMATQAFVLFQFPFTSCGTTRQITGDRAVYENELVATRDVKNGSRGSVTRD |
| | SIFRLHVSCSYSVSSNSLPINVQVFTLPPPFPETQPGPLTLELQIAKDKNYGSYYGVGDYPVVKLLRDP |
| | IYVEVSILHRTDPYLGLLLQQCWATPSTDPLSQPQWPILVKGCPYIGDNYQTQLIPVQKALDLPFPSHH |
| | QRFSIFTFSFVNPTVEKQALRGPVHLHCSVSVCQPAETPSCVVTCPDLSRRRNFDNSSQNTTASVSSKG |
| | PMILLQATKDPPEKLRVPVDSKVLWVAGLSGTLILGALLVSYLAVKKQKSCPDQMCQ |
| SEQ ID NO: 5 | QPLWLLQGGASHPETSVQPVLVECQEATLMVMVSKDLFGTGKLIRAADLTLGPEACEPLVSMDTEDVVR |
| | FEVGLHECGNSMQVTDDALVYSTFLLHDPRPVGNLSIVRTNRAEIPIECRYPRQGNVSSQAILPTWLPF |
| | RTTVFSEEKLTFSLRLMEENWNAEKRSPTFHLGDAAHLQAEIHTGSHVPLRLFVDHCVATPTPDQNASP |
| | YHTIVDFHGCLVDGLTDASSAFKVPRPGPDTLQFTVDVFHFANDSRNMIYITCHLKVTLAEQDPDELNK |
| | ACSFSKPSNSWFPVEGSADICQCCNKGDCGTPSHSRRQPHVMSQWSRSASRN |

1-12. (canceled)

13. A method of treating a human male subject by administering to the subject a pharmaceutical composition comprising:
a) a source of an immunogenic polypeptide comprising at least one of a class I MHC- and a class II MHC-restricted epitope being a portion of the amino acid sequence of a human Zona Pellucida protein (hZP) selected from hZP1, hZP2, hZP3, and hZP4, represented by SEQ ID NO: 1, SEQ ID NO: 2, SEQ ID NO: 3, and SEQ ID NO: 4, respectively;
b) a T cell comprising a T cell receptor that binds an MHC-peptide complex, wherein the peptide is a peptide from the amino acid sequence of an hZP selected from hZP1, hZP2, hZP3, and hZP4, represented by SEQ ID NO: 1, SEQ ID NO: 2, SEQ ID NO: 3, and SEQ ID NO: 4, respectively; or,
c) an antibody or fragment thereof that specifically binds to hZP selected from hZP1, hZP2, hZP3, and hZP4, represented by SEQ ID NO: 1, SEQ ID NO: 2, SEQ ID NO: 3, and SEQ ID NO: 4, wherein preferably, the hZP protein is hZP3 represented by SEQ ID NO: 3;
wherein the method is a contraceptive method, a method of reducing male fertility, a method of inducing male infertility, a method of inhibiting spermatogenesis, a method of inducing aspermia, a method of inducing azoospermia, a method of reducing sperm count, a method of inducing a state of severe oligospermia, a method of reducing Motile Total (MOT), a method of reducing total motile sperm (TMS) and/or a method of reducing semen volume.

14. The method of claim 13, wherein the source of an immunogenic polypeptide is a composition comprising: a1) one or more immunogenic peptides comprising or consisting of an amino acid sequence selected from SEQ ID NO.'s 66-75; and/or, a2) one or more immunogenic peptides comprising or consisting of an amino acid sequence selected from SEQ ID NO.'s 76-85.

15. The method of claim 13, wherein the source of the immunogenic polypeptide comprises at least one of:
a) a proteinaceous composition comprising at least one source of an immunogenic polypeptide comprising at least one of a class I MHC- and a class II MHC-restricted epitope being a portion of the amino acid sequence of a hZP selected from hZP1, hZP2, hZP3, and hZP4, represented by SEQ ID NO: 1, SEQ ID NO: 2, SEQ ID NO: 3, and SEQ ID NO: 4, respectively;
b) a nucleic acid molecule encoding an amino acid sequence of the immunogenic polypeptide;
c) a cell expressing the immunogenic polypeptide; and,
d) an antigen presenting cell presenting an MHC-peptide complex wherein the peptide is a peptide from the amino acid sequence of a hZP.

16. The method of claim 15, wherein:
a) the proteinaceous composition comprises at least one immunogenic polypeptide comprising a contiguous amino acid sequence of at least 18 amino acids selected from the amino acid sequence of a hZP selected from hZP1, hZP2, hZP3, and hZP4, represented by SEQ ID NO: 1, SEQ ID NO: 2, SEQ ID NO: 3, and SEQ ID NO: 4, respectively, and wherein the contiguous amino acid sequence comprises at least one of a class I MHC- and a class II MHC-restricted T cell epitope being a portion of the amino acid sequence of hZP selected from hZP1, hZP2, hZP3, and hZP4, represented by SEQ ID NO: 1, SEQ ID NO: 2, SEQ ID NO: 3, and SEQ ID NO: 4, respectively;
b) the nucleic acid molecule is: i) a DNA molecule that is an expression construct for expression of the immunogenic polypeptide in a human cell; or, ii) an RNA molecule that is capable of being translated into the immunogenic polypeptide in a human cell;
c) the cell is a microbial cell, preferably a Listeria cell; and, d) the antigen presenting cell is an autologous or allogeneic dendritic cell that is loaded ex vivo with an immunogenic polypeptide comprising at least one of a class I MHC- and a class II MHC-restricted epitope being a portion of the amino acid sequence of a hZP selected from hZP1, hZP2, hZP3, and hZP4, represented by SEQ ID NO: 1, SEQ ID NO: 2, SEQ ID NO: 3, and SEQ ID NO: 4, respectively.

17. The method of claim 16, wherein the proteinaceous composition comprises more than one different immunogenic polypeptides, each comprising a contiguous amino acid sequence of at least 18 amino acids selected from the amino acid sequence of a hZP selected from hZP1, hZP2, hZP3, and hZP4, represented by SEQ ID NO: 1, SEQ ID NO: 2, SEQ ID NO: 3, and SEQ ID NO: 4, respectively, and having a length in the range of 18-100 amino acids, preferably a length in the range of 18-60 amino acids.

18. The method of claim 13, wherein the hZP is an hZP3(23-350).

19. The method of claim 13, wherein the method further comprises administrating, preferably the co-administering, at least one adjuvant.

20. The method of claim 13, wherein the source of the one or more immunogenic peptides is messenger RNA that is capable of being translated into the immunogenic polypeptide in a human cell.

21. The method of claim 13, wherein the source of the one or more immunogenic peptides is lipid nanoparticle comprising the mRNA and a cationic lipid.

22. The method of claim 13, wherein the antibody or fragment thereof is a humanized or human monoclonal antibody or fragment thereof that specifically binds to hZP3(23-350).

23. The method of claim 13, wherein the effects of the method are reversible or transient.
